(19) United States
(12) Patent Application Publication
(10) Pub. No.: US 2025/0271139 A1
(43) Pub. Date: Aug. 28, 2025

(54) ONLINE SOFT MEASUREMENT METHOD FOR DIOXIN EMISSION CONCENTRATION IN MSWI PROCESS

(71) Applicant: BEIJING UNIVERSITY OF TECHNOLOGY, Beijing (CN)

(72) Inventors: Jian TANG, Beijing (CN); Heng XIA, Beijing (CN); Runyu ZHANG, Beijing (CN); Junfei QIAO, Beijing (CN)

(73) Assignee: BEIJING UNIVERSITY OF TECHNOLOGY, Beijing (CN)

(21) Appl. No.: 19/200,815

(22) Filed: May 7, 2025

Related U.S. Application Data
(63) Continuation of application No. PCT/CN2023/101309, filed on Jun. 20, 2023.

(30) Foreign Application Priority Data
Dec. 21, 2022 (CN) 202211651114.3

(51) Int. Cl. F23G 5/50; G06N 5/01

(52) U.S. Cl. CPC F23G 5/50; G06N 5/01; F23G 2208/00; F23G 2900/55

(57) ABSTRACT

An online soft measurement method for dioxin emission concentration in MSWI process includes: performing principal component analysis on process data according to a historical process data set of the MSWI process to obtain a drift index control limit; constructing an offline model based on FTBL, and inputting the process data and historical DXN true value data of the MSWI into an off-line model; performing principal component analysis according to online data, judging whether the online data is drift data according to the drift index control limit; if the typical sample pool is the drift data, constructing an online model based on FTBL, and inputting the process data and the drift data of the typical sample pool and output data of the incremental layer of the offline model into the online model; and determining a DXN emission concentration predicted value according to an offline calculation result and an online calculation result.

### CROSS REFERENCE TO THE RELATED APPLICATIONS

This application is the continuation application of International Application No. PCT/CN2023/101309, filed on Jun. 20, 2023, which is based upon and claims priority to Chinese Patent Application No. 202211651114.3, filed on Dec. 21, 2022, the entire contents of which are incorporated herein by reference.

### TECHNICAL FIELD

The invention relates to the technical field of pollutant monitoring, and in particular to an online soft measurement method for dioxin emission concentration in the MSWI process.

### BACKGROUND

Dioxin (DXN) is a persistent organic pollutant produced during municipal solid waste incineration (MSWI). It is an important environmental indicator that needs to be optimized and controlled to achieve the lowest emission, but it is difficult to monitor in real time because limited detection technology, economic, labor costs and other factors.

MSWI is one of the main technologies for realizing waste-to-energy generation and has been widely used around the world. As a typical industrial process for the detoxification, reduction and resource utilization of municipal solid waste (MSW), although the advantages of MSWI technology outweigh the disadvantages, the toxic and harmful substances contained in its emission gases have always been the focus of the public. DXN are the most toxic persistent organic pollutants known to the human body and the environment, and have become one of the factors restricting the development of MSWI technology. Due to the wide variety of compounds contained in DXN and the complex analytical procedures, long measurement delays, and high price, it is difficult to measure DXN emission concentration in real time. DXN is an important environmental indicator for the intelligent optimization control of the MSWI process. Therefore, the application of soft measurement technology is an effective means to solve the current high economic and labor cost consumption problems of DXN offline detection, and is also the basis for achieving ultra-low emissions of DXN.

At present, soft sensing technology mainly includes two strategies: mechanism model and data-driven model. Since the generation, adsorption and emission mechanisms of DXN are not yet clear, a mechanism model has not yet emerged. Data-driven models based on easily measured process variables are widely used due to their advantages of high efficiency, low cost, and ease of implementation. Therefore, this embodiment studies the construction of a soft measurement method for DXN emissions based on MSWI process data.

Due to the complexity of the mechanism of the incineration process, the volatility of MSW raw material components, and the randomness of manual control by experts in the field, the working condition drift phenomenon of the MSWI process frequently occurs. In addition, for practical engineering applications, it is difficult to establish a reliable soft measurement model of DXN emission concentration. Therefore, online soft measurement of DXN emissions needs to solve the following problems: In the MSWI process, the fluctuation of process variables measured by sensor data such as temperature, pressure, and flow is the main basis for characterizing changes in operating conditions. In the online application stage of the soft measurement model, how to identify drift changes in operating status based on process data to assist in accurate detection and model updating is one of the current challenges; how to maintain the ability to continuously and quickly learn new data (drift data) during the online measurement of DXN emission concentration is Key issues that need to be solved to realize practical engineering applications of soft sensing technology; making full use of historical data for offline modeling is the first step to achieve online soft sensing. How to select historical data to build an offline model with low cost and keep it optimal performance is the primary issue that needs to be solved in DXN soft sensor modeling.

### SUMMARY

In order to overcome the shortcomings of the existing technology, the purpose of the present invention is to provide an online soft measurement method for dioxin emission concentration in the MSWI process.

In order to achieve the above objects, the present invention provides the following solutions:

An online soft measurement method for dioxin emission concentration in the MSWI process, including:

Based on the K-means weighting algorithm, the process data of the typical sample pool is determined based on the historical process data set of MSWI;

Conduct principal component analysis based on the process data of the typical sample pool to obtain drift index control limits that reflect whether the MSWI process has changed;

Construct an offline model based on Fuzzy Tree-Based Learning (FTBL), and input the process data of the typical sample pool and the historical DXN true value data of MSWI into the offline model for prediction calculation to obtain offline calculation results; the offline model includes a feature mapping layer, enhancement and incremental layers;

Perform principal component analysis based on the obtained online data, and determine whether the online data is drift data or normal data based on the drift indicator control limit. If it is the normal data, jump to step “Building an offline FTBL-based model”, and input the process data of the typical sample pool and the historical DXN true value data of MSWI into the offline model for prediction calculation and obtain the calculation results; if it is the drift data, build an online model based on FTBL, and input the process data of the typical sample pool, the drift data and the output data of the incremental layer of the offline model into the online model for prediction calculation to obtain online calculation results; the online model includes online incremental layer;

The DXN emission concentration prediction value is determined based on the offline calculation result and the online calculation result.

Preferably, the process data for determining the typical sample pool based on the historical process data set of MSWI based on the K-means weighting algorithm includes:

Get the historical process data set X_{His} of MSWI;

Obtain historical data according to the historical D^{His}={xₙ, yₙ}ₙ ^{N}∈ [symbol] ^{N×(M+1)} according to the historical process data set X^{His};
where xₙ represent the n-th sample, yₙ represent the predict value of the n-th sample; N represent the number of sample in MSWI historical data set, M represent the feature number in MSWI historical data set;

Randomly select/instance as the initial centroid {Cᵢ}ᵢ₌₁^{I}.

All samples are listed into class/according to the weighted Euclidean distance between the sample and the centroid:

$${C}_{i} = {C}_{i} ⋃ min {\{ {d}_{n} \}}_{n = 1}^{N} = {C}_{i} ⋃ min {\{ \sqrt{{[ {x}_{n} - {C}_{i} ]}^{2} {w}_{T S}^{T}} \}}_{n = 1}^{N}$$

Among them, Cᵢ represents the i-th class; w_{TS}^{T} represents the weight vector of the process variable, where

$${w}_{TS}^{m} = H ( {x}_{m} ) - H ( {x}_{m} \vert y ) = {\sum }_{{x}_{n , m}} {\sum }_{{y}_{n}} p ( {x}_{n , m} , {y}_{n} ) {log}_{2} \frac{p ( {x}_{n , m} , {y}_{n} )}{p ( {x}_{n , m} ) p ( {y}_{n} )} ;$$

H(·) represents the information entropy of the random variable, xₘ is the m-th feature vector, y represents the DXN concentration, x_{n,m} represents the m-th eigenvalue of the n-th sample, p(x_{n,m}) and p(yₙ) represent the marginal probability distribution, and p(x_{n,m},yₙ) is the joint probability distribution;

Update the centroid Cᵢ using inter-class samples:

$${C}_{i} = \frac{1}{{N}_{{C}_{i}}} {\sum }_{x ∈ {C}_{i}} {x}_{n}$$

Among them, N_{C}_{ᵢ} represents the number of samples in the i-th cluster;

The centroids are updated cyclically, and all centroids are obtained through preset conditions, which are expressed as:

$${\{ {C}_{i} \}}_{i}^{I} ⇐ \vert {R}_{iter} - {R}_{iter - 1} \vert \leq {δ}^{TS}$$

where δ^{TS} is the threshold of the evaluation index Rᵢₜₑᵣ, iter represents the number of iterations, and the calculation formula of the measurement index is:

$${R}_{iter} = {\sum }_{i = 1}^{I} {{\sum }_{n}^{{N}_{{C}_{i}}} [ {x}_{n} - {C}_{i} ]}^{2} {W}_{TS}^{T}$$

TSP (Typical Sample Pool) is established by minimizing the clustering similarity, and the establishment formula is:

$${R}_{DB} = min ( \frac{1}{N} {\sum }_{n = 1}^{N} ( {S}_{i} + {S}_{j} ) / {M}_{ij} )$$

Among them, R_{DB} is the clustering similarity measure index;

$${S}_{i} = {\{ ( 1 / {N}_{{C}_{i}} ) \sum {\vert ( {x}_{n} - {C}_{i} ) {w}_{TS}^{T} \vert}^{a} \}}^{1 / a} {M}_{ij} = {\{ \sum {\vert {C}_{i} - {C}_{j} \vert}^{b} \}}^{1 / b}$$

where Sᵢ represents the sum of distances of the i-th category, and Mᵢⱼ represents the Minkowski metric criterion.

Preferably, principal component analysis is performed based on the process data of the typical sample pool to obtain drift indicator control limits that reflect whether the MSWI process has changed, including:

Express the correlation coefficient matrix of TSP data

$${D}^{TSP} = {\{ {x}_{n} , {y}_{n} \}}_{n}^{{N}_{TSP}} ∈ {ℝ}^{{N}_{TSP} ( M + 1 )} as R \approx {\frac{1}{{N}_{TSP} - 1} [ {X}^{TSP} ]}^{T} {X}^{TSP} ,$$

where, N_{TSP} is the number of TSP data D^{TSP}; R is the correlation coefficient matrix of TSP data;

Perform singular value decomposition on R and calculate eigenvalues; the calculation formula is R=U^{M×M}Σ^{M×M}[V^{M×M}]^{T}; where, U^{M×M} and V^{M×M} represent orthogonal matrices, and Σ^{M×M} is an M-dimensional pair angular matrix;

Use feature cumulative contribution rate η and Principal Component Analysis (PCA) contribution threshold δ^{PCA} for dimensionality reduction:

$$η = \frac{{\sum }_{{p}_{PCA} = 1}^{{P}_{PCA}} {σ}_{{p}_{PCA}}}{{\sum }_{m = 1}^{M} {σ}_{m}} .$$

Among them, P_{PCA} is the number of selected principal components, and P_{PCA} is less than M;

Rewrite the calculation formula as:

$$R = {U}^{M \times M} {\sum }^{M \times M} {V}^{M \times M} \approx {U}_{Key}^{{P}_{PCA} \times {P}_{PCA}} {{\sum }_{Key}^{{P}_{PCA} \times {P}_{PCA}} [ {U}_{Key}^{{P}_{PCA} \times {P}_{PCA}} ]}^{T}$$

where U_{Key}^{P}^{_{PCA}}^{×P}^{_{PCA}} is the load matrix;

According to the score matrix T and the load matrix U_{Key}^{P}^{_{PCA}}^{×P}^{_{PCA}}, X^{TPS} is expressed as:

$$\begin{matrix}{X}^{TSP} = {X}^{TSP} {{U}_{Key}^{{P}_{PCA} \times {P}_{PCA}} [ {U}_{Key}^{{P}_{PCA} \times {P}_{PCA}} ]}^{T} + \\ {X}^{TSP} ( {I}^{{P}_{PCA} \times {P}_{PCA}} - {{U}_{Key}^{{P}_{PCA} \times {P}_{PCA}} [ {U}_{Key}^{{P}_{PCA} \times {P}_{PCA}} ]}^{T} ) \\ = {T [ {U}_{Key}^{{P}_{PCA} \times {P}_{PCA}} ]}^{T} + {\tilde{T} [ {\tilde{U}}_{Key}^{{P}_{PCA} \times {P}_{PCA}} ]}^{T} \\ = {\hat{X}}^{TSP} + {\tilde{X}}^{TSP}\end{matrix}$$

Among them, \hat{X}^{TSP} represents the projection of X^{TSP} on the principal component space, \hat{X}^{TSP} represents the projection of \tilde{X}^{TSP} on the residual space and satisfy the orthogonal relationship; In addition, and satisfies the orthogonal relationship, which is proved as follows:

$$\begin{matrix}{[ {\hat{X}}^{TSP} ]}^{T} {\tilde{X}}^{TSP} = {[ {X}^{TSP} {{U}_{Key}^{{P}_{PCA} \times {P}_{PCA}} [ {U}_{Key}^{{P}_{PCA} \times {P}_{PCA}} ]}^{T} ]}^{T} \cdot {X}^{TSP} ( {I}^{{P}_{PCA} \times {P}_{PCA}} - \\ {{U}_{Key}^{{P}_{PCA} \times {P}_{PCA}} [ {U}_{Key}^{{P}_{PCA} \times {P}_{PCA}} ]}^{T} ) \\ = {{{U}_{Key}^{{P}_{PCA} \times {P}_{PCA}} [ {U}_{Key}^{{P}_{PCA} \times {P}_{PCA}} ]}^{T} [ {X}^{TSP} ]}^{T} {X}^{TSP} ( {I}^{{P}_{PCA} \times {P}_{PCA}} - \\ {{U}_{Key}^{{P}_{PCA} \times {P}_{PCA}} [ {U}_{Key}^{{P}_{PCA} \times {P}_{PCA}} ]}^{T} ) \\ = {{{U}_{Key}^{{P}_{PCA} \times {P}_{PCA}} [ {U}_{Key}^{{P}_{PCA} \times {P}_{PCA}} ]}^{T} [ {X}^{TSP} ]}^{T} {X}^{TSP} {I}^{{P}_{PCA} \times {P}_{PCA}} - \\ {{{U}_{Key}^{{P}_{PCA} \times {P}_{PCA}} [ {U}_{Key}^{{P}_{PCA} \times {P}_{PCA}} ]}^{T} [ {X}^{TSP} ]}^{T} \\ {X}^{TSP} {{U}_{Key}^{{P}_{PCA} \times {P}_{PCA}} [ {U}_{Key}^{{P}_{PCA} \times {P}_{PCA}} ]}^{T}\end{matrix}$$

$$subject to {{U}_{Key}^{{P}_{PCA} \times {P}_{PCA}} [ {U}_{Key}^{{P}_{PCA} \times {P}_{PCA}} ]}^{T} = {I}^{{P}_{PCA} \times {P}_{PCA}} = {[ {X}^{TSP} ]}^{T} {X}^{TSP} - {[ {X}^{TSP} ]}^{T} {X}^{TSP} = 0$$

The drift indicator control limit is expressed as:

$${T}_{CL}^{2} = \frac{{P}_{PCA} ( {N}_{TSP} - 1 )}{( {N}_{TSP} - {P}_{PCA} )} {F}_{α} ( {P}_{P C A} , {N}_{T S P} - {P}_{P C A} ) ;$$

$${SPE}_{CL} = {{Θ}_{1} ( \frac{{c}_{α} \sqrt{2 {Θ}_{2} {h}_{0}^{2}}}{{Θ}_{1}} + 1 + \frac{{Θ}_{2} {h}_{0} ( {h}_{0} - 1 )}{{Θ}_{1}^{2}} )}^{1 / {h}_{0}} ;$$

Among them, T_{CL}² is the control limit of Hotelling's T²; SPE_{CL} is the control limit of Squared Prediction Error (SPE); P_{PCA} is the number of selected principal components F_{α}(P_{PCA}, N_{TSP}−P_{PCA}), F_{α}(P_{PCA}, N_{TSP}−P_{PCA}) represents the F distribution with degrees of freedom of P_{PCA} and (N_{TSP}−P_{PCA}); c_{α} represents not normal deviation exceeding (1−α); Θ₁, Θ₂ and h₀ are calculated as follows:

$${h}_{0} = 1 - 2 {Θ}_{1} {Θ}_{3} / 3 {Θ}_{1}^{2} ;$$

$${Θ}_{i} = {\sum }_{m = {P}_{PCA} + 1}^{M} {( {σ}_{m} )}^{i} , i = 1 , 2 , 3 ;$$

Among them, Θ₁, Θ₂ and h₀, are all intermediate variables in SPE control limit calculation, and σₘ is the eigenvalue of singular value decomposition.

Preferably, an offline model based on FTBL is constructed, and the process data of the typical sample pool and the historical DXN true value data of MSWI are input into the offline model for prediction calculation, and the offline calculation results are obtained, including:

For the given TSP data D^{TSP}={xₙ, yₙ}ₙ ^{N}^{_{TSP}}∈ [symbol] ^{N}^{_{TSP}}^{x(M+1)}, randomly select a feature value x in D^{TSP} to define node splitting function μ_{CS}^{t}(x), where:
μ_{CS}^{t}(x)=rand(n,m), n∈(1, N_{TSP}) and m∈(1,M)
   where μ_{CS}(x) is a symbolic function, rand(·) is a random number generation function, n and m do not take the maximum and minimum values;

Determine K fuzzy rules for TS fuzzy reasoning, and the k-th rule can be expressed as:

$${R}_{k} : if {x}_{1}^{{t}_{leaf}} is {φ}_{1}^{k} ( {x}_{1}^{{t}_{leaf}} ) and … and {x}_{{M}^{{t}_{leaf}}}^{{t}_{leaf}} is {μ}_{{M}^{{t}_{leaf}}}^{k} ( {x}_{{M}^{{t}_{leaf}}}^{{t}_{leaf}} )$$

$$then {y}^{k} = {g}^{k} ( {x}_{1} , … , {x}_{{M}^{{t}_{leaf}}} )$$

Wherein:

$${φ}_{m}^{k} ( {x}_{m}^{{t}_{leaf}} ) = exp [ - {( {x}_{m}^{{t}_{leaf}} - {c}_{k , m} )}^{2} / {σ}_{k , m}^{2} ] ;$$

Among them, Rₖ is the k-th fuzzy rule, c_{k,m} and σ_{k,m} respectively represent the center and width of the Gaussian function φₘ^{k}(·), t_{leaf} represents the t_{leaf}-th leaf node; φₘ^{k}(·) is the Gaussian function;

According to the above K fuzzy rules, the result of Fuzzy Decision Tree (FDT) is described as:

$${\hat{y}}_{i} = f ( {x}_{i} ) = {\sum }_{k = 1}^{K} {\overline{o}}^{k} {g}^{k} ( {x}_{i} )$$

Wherein:

$${\overline{o}}^{k} = {\prod }_{m = 1}^{{M}^{Leaf}} {φ}_{m}^{k} ( {x}_{m}^{{t}_{leaf}} ) / {\sum }_{i = 1}^{K} ( {\prod }_{m = 1}^{{M}^{Leaf}} {φ}_{m}^{k} ( {x}_{m}^{{t}_{leaf}} ) )$$

$${g}^{k} ( {x}_{i}^{Leaf} ) = {x}_{i}^{Leaf} {ω}_{TS}^{k}$$

Among them, f(·) is the FDT model, ō^{k} and g^{k}(·) represents the antecedent and consequent parts of TS fuzzy reasoning, and ω_{TS}^{k} represents the weight of the features of the consequent part;

Apply the gradient descent method to update the parameters during the training process of the FDT model f(·). The parameters include the center cₖ, the width σₖ and the weight ω_{TS}^{k}. The output of the feature mapping layer is expressed as follows:

$${Z}_{FM}^{{N}_{FM}} = [ {z}_{FM}^{1} , {z}_{FM}^{2} , … , {z}_{FM}^{{N}_{FM}} ] ∈ {ℝ}^{{N}_{TSP} \times {N}_{FM}}$$

$$where :$$

$${z}_{FM}^{{N}_{FM}} = {f}_{{n}_{FM}}^{FM} ( {X}^{TSP} ) ∈ {ℝ}^{{N}_{TSP} \times 1}$$

where, z_{FM}ⁿ^{_{PM}} is the output of the n_{FM}-th FDT model through the input X^{TSP}.

The enhancement layer takes Z_{FM}^{N}^{_{FM}} as input, and the output of the enhancement layer is expressed as:

$${Z}_{En}^{{N}_{EN}} = [ {z}_{En}^{1} , {z}_{En}^{2} , … , {z}_{En}^{{N}_{En}} ] ∈ {ℝ}^{{N}_{TSP} \times {N}_{En}}$$

The output of the feature mapping layer and the enhancement layer is

$${G}^{{N}_{FM} + {N}_{EN}} = [ {Z}_{FM}^{{N}_{FM}} \vert {Z}_{En}^{{N}_{EN}} ]$$

Use the ridge regression learning algorithm to calculate the weight W^{N}^{_{FM}}^{+N}^{_{En}} between G^{N}^{_{FM}}^{+N}^{_{En}} and the predicted output:

$$\begin{matrix}{W}^{{N}_{FM} + {N}_{En}} = {( λ I + {{G}^{{N}_{FM} + {N}_{En}} ( {G}^{{N}_{FM} + {N}_{En}} )}^{T} )}^{- 1} {( {G}^{{N}_{FM} + {N}_{En}} )}^{T} {y}^{TSP} \\ = ( {G}^{{N}_{FM} + {N}_{En}} ) * {y}^{TSP}\end{matrix}$$

Among them, (G^{N}^{_{FM}}^{+N}^{_{En}})* is the pseudo-inverse matrix, λ is the regularization coefficient, and I is the identity matrix;

Add the FDT model in the incremental layer and dynamically update the pseudo-inverse matrix, taking Z_{En}^{N}^{_{En}} as input and G^{N}^{_{FM}}^{+N}^{_{En}}⁺¹=[Z_{FM}^{N}^{_{FM}}|Z_{En}^{N}^{_{En}}|z_{In}¹] as output; the pseudo-inverse matrix update process of the incremental process is as follows:

$${[ {G}^{{N}_{FM} + {N}_{En} + 1} ]}^{*} = [ \begin{matrix}{[ {G}^{{N}_{FM} + {N}_{En} + 1} ]}^{*} - D {B}^{T} \\ {B}^{T}\end{matrix} ]$$

$$Wherein :$$

$$\{ \begin{matrix}D = {[ {G}^{{N}_{FM} + {N}_{En} + 1} ]}^{*} {H}_{k + 1} \\ {B}^{T} = \{ \begin{matrix}{[ C ]}^{*} , \\ {[ 1 + {D}^{T} D ]}^{- 1} {{D}^{T} [ {G}^{{N}_{FM} + {N}_{En} + 1} ]}^{*}\end{matrix} \\ C = {H}_{k + 1} - {G}^{{N}_{FM} + {N}_{En} + 1} D\end{matrix} ; \begin{matrix}if C \neq 0 \\ if C = 0\end{matrix}$$

Among them, D Hₖ₊₁, B^{T} and C are all intermediate variables in the pseudo-inverse matrix update process;

The new weight matrix W^{N}^{_{FM}}^{+N}^{_{En}}⁺¹ is expressed as:

$${W}^{{N}_{FM} + {N}_{En} + 1} = [ \begin{matrix}{W}^{{N}_{FM} + {N}_{En}} - {DB}^{T} {y}^{TSP} \\ {B}^{T} {y}^{TSP}\end{matrix} ]$$

The prediction calculation process of the offline model FTBL is as follows:

$$\hat{y} = {G}^{{N}_{FM} + {N}_{En} + 1} {W}^{{N}_{FM} + {N}_{En} + 1}$$

Preferably, principal component analysis is performed based on the obtained online data, and whether the online data is judged to be drift data or normal data based on the drift indicator control limit, if it is the normal data, jump to step “Construct an offline model based on FTBL, and input the process data of the typical sample pool and the historical DXN true value data of MSWI into the offline model for prediction calculation and obtain the calculation result”; if it is the drift data, construct Based on the FTBL online model, the process data of the typical sample pool, the drift data and the output data of the incremental layer of the offline model are input into the online model for prediction calculation, and the online calculation results are obtained, including:

Calculate the drift value within the new window based on:

$${T}_{{O}_{1}}^{2} = {x}_{{O}_{1}}^{T} {U}_{{N}_{TSP} + 1}^{{P}_{PCA} \times {P}_{PCA}} {\sum }_{{N}_{TSP} + 1}^{- 1} {( {U}_{{N}_{TSP} + 1}^{{P}_{PCA} \times {P}_{PCA}} )}^{T} {x}_{{O}_{1}} ;$$

$${SPE}_{{O}_{1}} = {x}_{{N}_{TSP} + 1}^{T} ( I - {{U}_{{N}_{TSP} + 1}^{{P}_{PCA} \times {P}_{PCA}} ( {U}_{{N}_{TSP} + 1}^{{P}_{PCA} \times {P}_{PCA}} )}^{T} ) {x}_{{N}_{TSP} + 1} ;$$

Among them, T_{O}_{₁}² and SPE_{O}_{₁} are the statistical indicators of the (N_{TSP}+1)-th process data U_{N}_{_{TSP}}₊₁^{P}^{_{PCA}}^{×P}^{_{PCA}} represent the new load matrix, Σ_{N}_{_{TSP}}₊₁ represent the new diagonal matrix;

Determine whether the sample is a drift sample or a normal sample through a judgment formula; the judgment formula is:

$$\{ \begin{matrix}drift & if {T}_{{O}_{1}}^{2} > {T}_{CL}^{2} and {SPE}_{{O}_{1}} > {SPE}_{CL} \\ normal & etc .\end{matrix}$$

For normal samples, the offline model of FTBL is reused for soft measurement of DXN concentration, which can be expressed as:

$${\hat{y}}_{{O}_{t}} = {G}^{k + {N}_{In}} {W}^{k + In} = [ {Z}_{FM}^{{N}_{FM}} \vert {Z}_{En}^{{N}_{EN}} \vert {Z}_{In}^{{N}_{In}} ] {W}^{k + {N}_{In}} = [ {f}_{1}^{FM} ( {x}_{{O}_{t}} ) , … , {f}_{{N}_{FM}}^{FM} ( {x}_{{O}_{t}} ) \vert {f}_{1}^{En} ( {x}_{{O}_{t}} ) , … , {f}_{{N}_{En}}^{En} ( {x}_{{O}_{t}} ) \vert {f}_{1}^{In} ( {x}_{{O}_{t}} ) , … , {f}_{{N}_{In}}^{In} ( {x}_{{O}_{t}} ) ] {W}^{k + {N}_{In}}$$

For drift samples, the soft measurement value is calculated as:

$${\hat{y}}_{{O}_{t}} = {G}^{k + {N}_{In}} {W}^{k + In} + {ε}_{Offset}$$

Among them, ε_{Offset} is the offset value of offline FTBL prediction output is as follows:

$${ε}_{Offset} = \{ \begin{matrix}+ \frac{1}{{N}_{t}} \sum ( {y}_{t} - E ( \hat{y} ) ) , if {x}_{IncTem} < E ( {x}_{IncTem} ) \\ - \frac{1}{{N}_{t}} \sum ( {y}_{t} - E ( \hat{y} ) ) , if {x}_{IncTem} \geq E ( {x}_{IncTem} )\end{matrix}$$

Among them, Nₜ represents the total amount of data arriving at time t, ŷ represents all the predicted values arriving at time t, E(ŷ) represents the mathematical expectation of the vector ŷ, and X_{IncTem} represents the incinerator temperature at time t.

When the true value is detected, the TSP data, drift data and the output of the incremental layer are input into the online model; the predicted value of the online model is:

$${\hat{y}}_{{O}_{t}} = {G}^{{N}_{FM} + {N}_{En} + {N}_{In} + {N}_{OI}} {W}^{{N}_{FM} + {N}_{En} + {N}_{In} + {N}_{OI}}$$

Among them, W^{N}^{_{FM}}^{+N}^{_{En}}^{+N}^{_{In}}^{+N}^{_{OI}} represents the weight matrix, and G^{N}^{_{FM}}^{+N}^{_{En}}^{+N}^{_{In}}^{+N}^{_{OI}} is the FDT output matrix of N_{FM}+N_{En}+N_{In}+N_{OI}.

According to the specific embodiments provided by the present invention, the present invention discloses the following technical effects:

The invention provides an online soft measurement method for MSWI process dioxin emission concentration, which is characterized in that it includes: based on the K-means weighting algorithm, determining the process data of a typical sample pool according to the historical process data set of MSWI; Conduct principal component analysis on the process data of the sample pool to obtain drift indicator control limits that reflect whether the MSWI process has changed; construct an offline model based on FTBL, and input the process data of the typical sample pool and the historical DXN true value data of MSWI into prediction calculations are performed in the offline model to obtain offline calculation results; the offline model includes a feature mapping layer, an enhancement layer and an incremental layer; principal component analysis is performed based on the obtained online data, and judgment is made based on the drift indicator control limit whether the online data is drift data or normal data, if it is the normal data, jump to step “Build an offline model based on FTBL, and input the process data of the typical sample pool and the historical DXN true value data of MSWI into the offline model for prediction calculation and obtain the calculation result”; if it is the drift data, construct an online model based on FTBL, and input the process data of the typical sample pool, the drift data and the offline output data of the incremental layer of the model into the online model for prediction calculation to obtain online calculation results; the online model includes an online incremental layer; the predictive value of DXN emission concentration is determined based on the offline calculation results and the online calculation results. The present invention can effectively improve the accuracy of DXN emission concentration prediction values.

### BRIEF DESCRIPTION OF THE DRAWINGS

In order to more clearly illustrate the embodiments of the present invention or the technical solutions in the prior art, the drawings needed to be used in the embodiments will be briefly introduced below. Obviously, the drawings in the following description are only some of the present invention. Embodiments, for those of ordinary skill in the art, other drawings can also be obtained based on these drawings without exerting creative efforts.

FIG. 1 is a schematic diagram of the MSWI process and DXN emission provided by the embodiment of the present invention;

FIG. 2 is a schematic diagram of the DXN concentration measurement process provided by the embodiment of the present invention;

FIG. 3 is a method flow chart provided by an embodiment of the present invention;

FIG. 4 is a schematic diagram of the DXN concentration soft measurement strategy provided by the embodiment of the present invention;

FIG. 5 is a schematic diagram of a fuzzy decision tree provided by an embodiment of the present invention;

FIG. 6 is a first schematic diagram of DXN data provided by an embodiment of the present invention;

FIG. 7 is a second schematic diagram of DXN data provided by an embodiment of the present invention;

FIG. 8 is a schematic diagram of training data fitting curves of different methods provided by embodiments of the present invention;

FIG. 9 is a schematic diagram of test data fitting curves of different methods provided by the embodiment of the present invention;

FIG. 10 is a schematic three-dimensional curve diagram of a typical sample provided by an embodiment of the present invention;

FIG. 11 is a schematic diagram of a two-dimensional curve of a typical sample provided by an embodiment of the present invention;

FIG. 12 is a schematic diagram of a three-dimensional curve of a typical sample with redundant samples deleted according to an embodiment of the present invention;

FIG. 13 is a schematic diagram of the T² curve provided by the embodiment of the present invention;

FIG. 14 is a schematic diagram of the SPE curve provided by the embodiment of the present invention;

FIG. 15 is a schematic diagram of the offline stage prediction results provided by the embodiment of the present invention;

FIG. 16 is a schematic diagram of the online stage prediction results provided by the embodiment of the present invention;

FIG. 17 is a schematic diagram of laboratory simulation application test provided by the embodiment of the present invention;

FIG. 18 is a schematic diagram of an industrial field online application test provided by an embodiment of the present invention;

### DETAILED DESCRIPTION OF THE EMBODIMENTS

The technical solutions in the embodiments of the present invention will be clearly and completely described below with reference to the accompanying drawings in the embodiments of the present invention. Obviously, the described embodiments are only some of the embodiments of the present invention, rather than all the embodiments. Based on the embodiments of the present invention, all other embodiments obtained by those of ordinary skill in the art without creative efforts fall within the scope of protection of the present invention.

Reference in this embodiment to “an embodiment” means that a particular feature, structure or characteristic described in connection with the embodiment can be included in at least one embodiment of the present application. The appearances of this phrase in various places in the specification are not necessarily all referring to the same embodiment, nor are separate or alternative embodiments mutually exclusive of other embodiments. Those skilled in the art understand explicitly and implicitly that the embodiments described in this embodiment can be combined with other embodiments.

The terms “first”, “second”, “third” and “fourth” in the description, claims and drawings of this application are used to distinguish different objects, rather than to describe a specific sequence. Furthermore, the terms “including” and “having” and any variations thereof are intended to cover non-exclusive inclusion. For example, a series of steps, processes, methods, etc. are not limited to the listed steps, but optionally also include steps that are not listed, or optionally also include steps inherent to these processes, methods, products or equipment. Other steps.

The purpose of the present invention is to provide an online soft measurement method for dioxin emission concentration in the MSWI process, which can effectively improve the accuracy of the DXN emission concentration prediction value.

In order to make the above objects, features and advantages of the present invention more obvious and understandable, the present invention will be described in further detail below with reference to the accompanying drawings and specific embodiments.

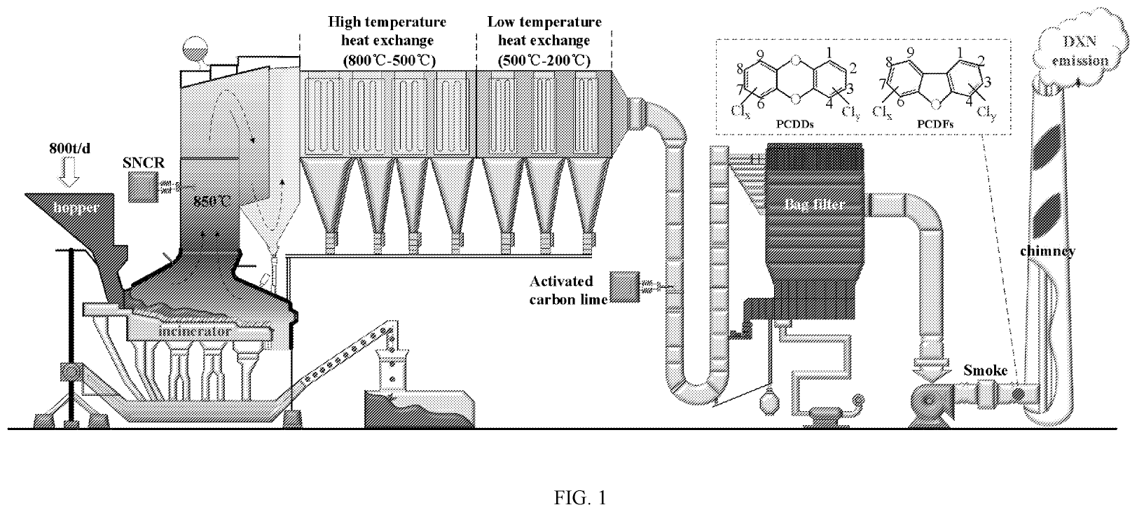

As shown in FIG. 1, this embodiment divides the MSWI process into five stages:
(1) MSW is fed into the hopper through a mechanical grab, and then transported to the incinerator through a moving grate;
(2) In a high-temperature environment above 850° C., MSW undergoes drying, pyrolysis and gasification stages on the grate to form ash;
(3) The thermal energy of high-temperature flue gas is converted into high-temperature and high-pressure steam through heat exchange devices such as water-cooled walls, superheaters, evaporators, economizers, and steam drums. The steam is then used to drive the turbine generator to convert waste into energy;
(4) Selective non-catalytic reduction (SNCR) system denitrification, activated carbon adsorption, semi-dry deacidification, bag dust collector, etc. are used to remove toxic and harmful substances (such as CO, HCl, SO₂, NOₓ, particulate matter, DXN) produced by the MSWI process;
(5) Flue gas that meets the pollutant control emission standards (GB18485-2014) is discharged into the atmosphere through an 80-meter-high chimney.

Research shows that native MSW contains trace amounts of DXN. During the combustion and purification process of MSW, DXN undergoes high-temperature decomposition, low-temperature de novo synthesis, activated carbon adsorption, and bag dust removal, and the remaining trace amounts of DXN are emitted into the atmosphere. In addition, DXN has a significant “memory effect” due to the accumulation of fly ash in MSWI equipment. Therefore, applying soft sensing methods to detect DXN emission concentration is a challenging task. Usually, the DXN emission concentration detection position of the MSWI process is located at the chimney inlet (that is, the position marked by the dot where the flue gas intersects with the chimney in FIG. 1).

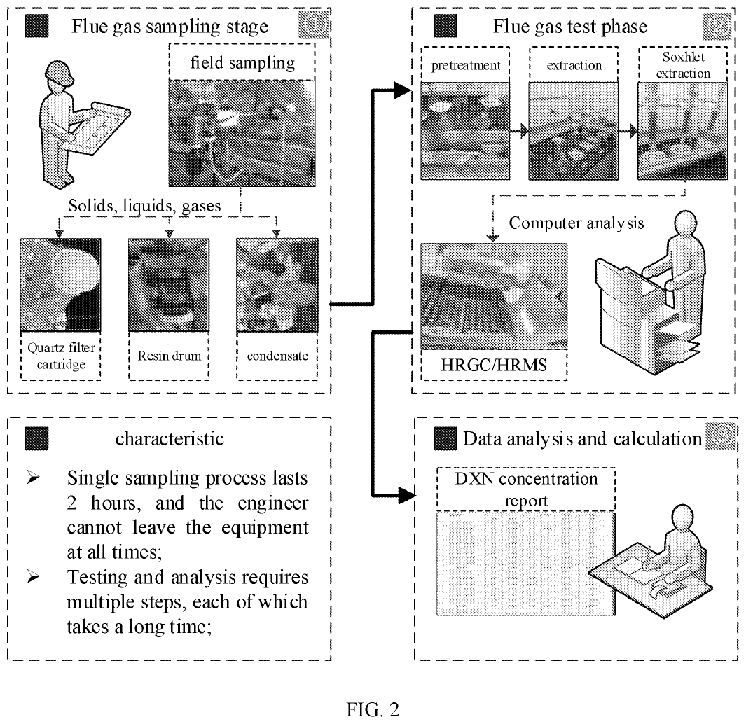

To date, detection of DXN emission concentrations has mainly relied on manual on-site sampling and laboratory analysis. The detection process is shown in FIG. 2.

In FIG. 2, the first stage is manual on-site sampling by field engineers. It requires using a constant speed sampling device to continuously collect real-time flue gas in the pipeline for two hours to obtain quartz filter cartridge (solid phase), resin cartridge (gas phase) and condensate (liquid phase), and then packaged and sent to the laboratory for analysis; the second stage is sample analysis. These samples need to be pretreated, extracted and Soxhlet extracted, and then put into designated reagent bottles, and then used high-resolution gas chromatography-high resolution mass spectrometry (HRGC/HRMS) equipment to analyze the sample; the third stage is to analyze and calculate the HRGC/HRMS data and obtain a DXN concentration report containing 17 compounds.

DXN concentration detection has the disadvantages of long time consumption, high labor and material costs, and the inability to continuously and timely reflect the DXN emissions from the incinerator. At the same time, the DXN modeling data obtained based on the above experiments has the characteristics of small sample size and high dimensionality. In addition, due to the unclear mechanism of DXN and many human interference factors in the detection process, it is difficult for soft measurement technology based on actual DXN data to achieve satisfactory detection accuracy. Therefore, it is a huge challenge to study the soft measurement model of DXN emission concentration with stable and high performance.

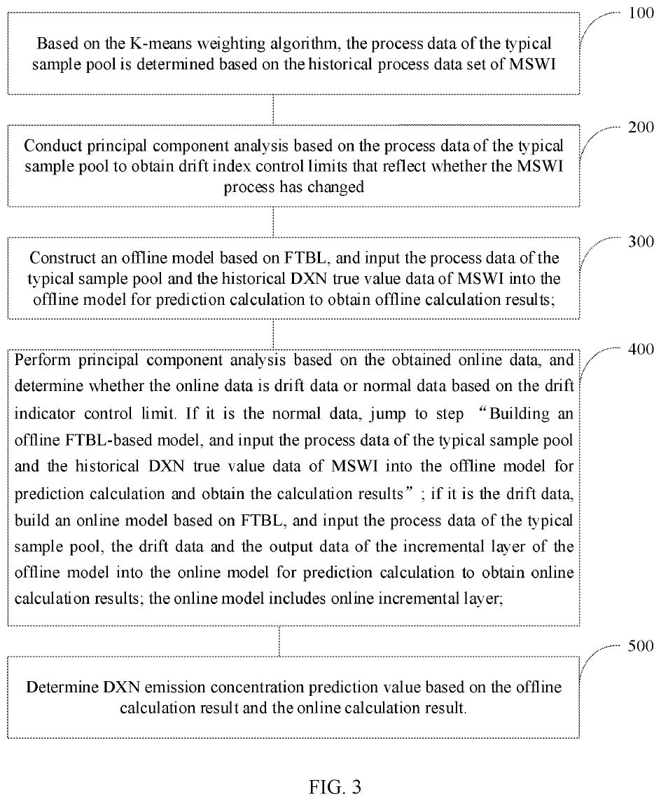

FIG. 3 shows an online soft measurement method for dioxin emission concentration in the MSWI process provided by this embodiment, including:
Step 100: Based on the K-means weighting algorithm, determine the process data of the typical sample pool based on the historical process data set of MSWI;
Step 200: Perform principal component analysis based on the process data of the typical sample pool to obtain drift index control limits that reflect whether the MSWI process has changed;
Step 300: Construct an offline model based on FTBL, and input the process data of the typical sample pool and the historical DXN true value data of MSWI into the offline model for prediction calculation to obtain offline calculation results; the offline model includes features mapping layer, enhancement layer and incremental layer; Step 400: Perform principal component analysis based on the acquired online data, and determine whether the online data is drift data or normal data based on the drift indicator control limit. If it is the normal data, jump to step “Constructing a data based on the offline model of FTBL, and input the process data of the typical sample pool and the historical DXN true value data of MSWI into the offline model for prediction calculation”, and obtain the calculation result ”; if it is the drift data, construct an online model based on FTBL, and input the process data of the typical sample pool, the drift data and the output data of the incremental layer of the offline model into the online model for prediction calculation to obtain online calculation results; the online model includes an online incremental layer;
Step 500: Determine the DXN emission concentration prediction value according to the offline calculation result and the online calculation result.

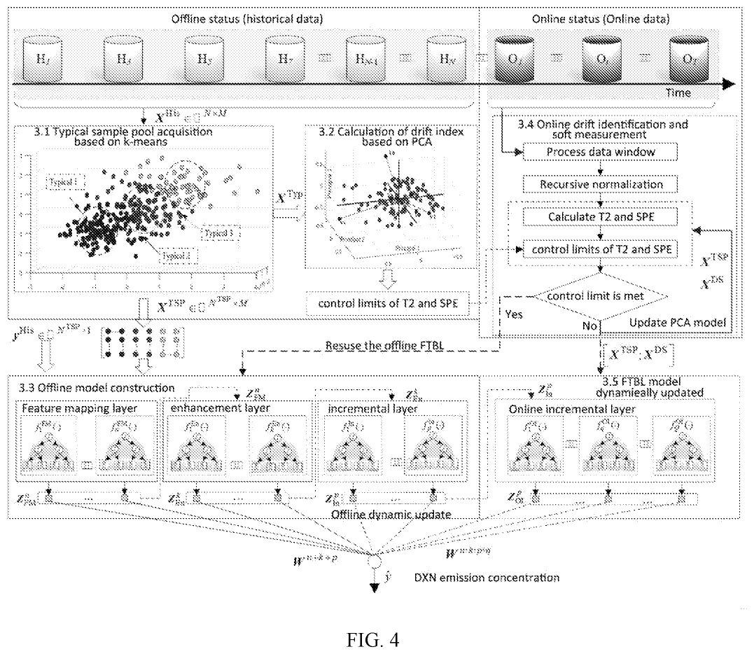

The DXN emission concentration soft measurement structure proposed in this embodiment is shown in FIG. 4, including two stages: offline and online. In the offline stage, TSP, FTBL algorithm and PCA analysis are used to obtain the offline model and drift indicator control limits based on historical process data. In the online stage, the sliding window recursive PCA adaptive monitoring process is used to implement drift detection, online measurement and FTBL dynamic learning. In FIG. 4, the meanings of different symbols are shown in Table 1 below.

TABLE 1

| serial | | |
|---|---|---|
| num- | | |
| ber | symbol | implication |
| 1 | H_{N} | N-th historical process data of MSWI |
| 2 | O_{T} | MSWI's online process data at time T |
| 3 | X^{His} | Historical process data set for MSWI |
| 4 | N | The number of samples of MSWI's |
| | | historical data set |
| 5 | M | The number of sfeatures of MSWI's |
| | | historical data set |
| 6 | X^{TSP} | Process data of a typical sample pool |
| 7 | N^{TSP} | Number of samples in a typical |
| | | sample pool |
| 8 | y^{His} | Historical DXN truth data of MSWI |
| 9 | ƒᵢ^{FM}(·), i = 1···n | FDT model in the feature |
| | | mapping layer |
| 10 | Z_{FM}ⁿ | Output matrix of the feature |
| | | mapping layer |
| 11 | ƒₗ^{En}(·), i = 1···k | FDT model in the enhancement layer |
| 12 | Zₑₙ^{k} | Output matrix of the |
| | | enhancement layer |
| 13 | ƒₗ^{In}(·), i = 1···p | FDT model in the Incremental layer |
| 14 | Z_{In}^{p} | Output matrix of the Incremental layer |
| 15 | X^{DS} | Drift sample set in online window |
| 16 | ƒₗ^{OI}(·), i = 1···Q | Function of the FDT model in the |
| | | online increment layer |
| 17 | Z_{OI}^{p} | The output matrix of the online |
| | | increment layer |
| 18 | W^{n+k+p} | weight matrix |
| 19 | ŷ | DXN emission concentration |
| | | prediction |
| 20 | D^{His} | historical data |
| 21 | Cᵢ | i-th centroid |
| 22 | xₙ | n-th sample |
| 23 | W_{TS}^{T} | The weight of the process variable |
| 24 | dₙ | weighted euclidean distance |
| 25 | H(·) | Entropy of a random variable |
| 26 | xₘ | m-th eigenvector |
| 27 | x_{n,m} | The m-th eigenvalue of the n-th sample |
| 28 | p(·) | Marginal probability distribution |
| 29 | p(·,·) | joint probability distribution |
| 30 | N_{C}_{ᵢ} | The number of the i-th sample |
| | | in the cluster |
| 31 | Rᵢₜₑᵣ | Sum of Euclidean distances |
| | | between the cluster sample |
| | | and the centroid |
| 32 | δ^{TS} | The threshold of the evaluation |
| | | indicator Rᵢₜₑᵣ |
| 33 | R_{DB} | Clustering similarity measure index |
| 34 | Sᵢ | Sum of distances of class i |
| 35 | Mᵢⱼ | Minkowski metric criteria |
| 36 | D^{TSP} | Typical sample pool data |
| 37 | N_{TSP} | The number of samples in D^{TSP} |
| 38 | R | Correlation coefficient matrix |
| | | of TSP data |
| 39 | U^{MxM} | Orthogonal matrix of singular |
| | | value decomposition |
| 40 | V^{MxM} | Orthogonal matrix of singular |
| | | value decomposition |
| 41 | σₘ | Eigenvalues of singular value |
| | | decomposition |
| 42 | η | Characteristic cumulative |
| | | contribution rate |
| 43 | δ_{PCA} | Contribution threshold of PCA |
| 44 | P_{PCA} | The number of selected principal |
| | | components |
| 45 | T | Score matrix |
| 46 | U_{Key}^{P}^{_{PCA}}^{xP}^{_{PCA}} | Load matrix |
| 47 | \hat{X}^{TSP} | Projection of X^{TPS} onto the principal |
| | | component space |
| 48 | \tilde{X}^{TSP} | Projection of X^{TPS} on the residual space |
| 49 | T_{CL}² | Control limit of Hotelling's T² |
| 50 | SPE_{CL} | Control limit of SPE |
| 51 | F_{α}(P_{PCA}, | F distribution with a freedom degree |
| | N_{TSP} − P_{PCA}) | of P_{PCA} and (N_{TSP} − P_{PCA}) |
| 52 | c_{α} | A normal deviation of not more |
| | | than (1 − α) |
| 53 | Θ₁ | Intermediate variables that the SPE |
| | | controls limit computation |
| 54 | Θ₂ | Intermediate variables that the |
| | | SPE controls limit computation |
| 55 | h₀ | Intermediate variables that the SPE |
| | | controls limit computation |
| 56 | μ′_{cs}(x) | Node splitting function |
| | | (symbolic function) |
| 57 | rand(·) | Random number generation function |
| 58 | D^{Leaf} | Sample set of leaf nodes |
| 59 | xₙ^{Leaf} | The process data in the sample |
| | | set in the node |
| 60 | Rₖ | k-th rule of ambiguity |
| 61 | φₘ^{k}(·) | Gaussian function |
| 62 | c_{k,m} | Center of Gaussian function φₘ^{k}(·) |
| 63 | σ_{k,m} | Width of Gaussian function φₘ^{k}(·) |
| 64 | t_{leaf} | t_{leaf}-th leaf node |
| 65 | ō^{k} | Antecedent part of TS fuzzy reasoning |
| 66 | g^{k}(·) | Consequent part of TS fuzzy reasoning |
| 67 | ω_{TS}^{k} | The weights of the features |
| | | of the consequent part of |
| | | TS fuzzy reasoning |
| 68 | Z_{FM}^{N}^{_{FM}} | The output of feature mapping layer |
| 69 | Z_{FM}ⁿ^{_{FM}} | The output of the n_{FM}-th FDT model |
| 70 | Z_{En}^{N}^{_{En}} | The output of enhancement layer |
| 71 | G^{N}^{_{FM}}^{+N}^{_{En}} | Output of feature mapping layer |
| | | and enhancement layer |
| 72 | W^{N}^{_{FM}}^{+N}^{_{En}} | The weight between G^{N}^{_{FM}}^{+ N}^{_{En}} and |
| | | the predicted output |
| 73 | λ | Regularization coefficient of |
| | | ridge regression |
| 74 | (G^{N}^{_{FM}} ⁺ ^{N}^{_{En}})* | Pseudo-inverse matrix |
| 75 | I | Unit matrix |
| 76 | G^{N}^{_{FM}}^{+N}^{_{En}}⁺¹ | The output of feature mapping |
| | | layer, enhancement layer |
| | | and Incremental layer |
| 77 | W^{N}^{_{FM}}^{+N}^{_{En}}⁺¹ | The weight between G^{N}^{_{FM}}^{+N}^{_{En}}⁺¹ |
| | | and the predicted output |
| 78 | [G^{N}^{_{FM}}^{+N}^{_{En}}⁺¹]* | Pseudo-inverse matrix |
| 79 | D | Intermediate variable of the |
| | | pseudo-inverse matrix |
| | | update process |
| 80 | Hₖ₊₁ | Intermediate variable of the |
| | | pseudo-inverse matrix |
| | | update process |
| 81 | B^{T} | Intermediate variable of the |
| | | pseudo-inverse matrix |
| | | update process |
| 82 | C | Intermediate variable of the |
| | | pseudo-inverse matrix |
| | | update process |
| 83 | T₀_{ₗ}² | T2 statistical index of the |
| | | (N_{TSP} + 1)-th process data(x₀_{ₗ}) |
| 84 | SPE₀_{ₗ} | SPE statistical index of the |
| | | (N_{TSP} + 1)-th process data(x₀_{ₗ}) |
| 85 | U_{N}_{_{TSP}}₊₁^{P}^{_{PCA}}^{xP}^{_{PCA}} | New load matrix |
| 86 | Σ_{T}_{_{SP}}₊₁ | Diagonal matrix |
| | | (diag(σ₁, σ₂, . . . , σ_{PCA})) |
| 87 | ŷₒ_{ₜ} | The predicted value of the MSWI |
| | | process data at time t |
| 88 | ε_{Offset} | The offset value of the offline FTBL |
| | | predicts output |
| 89 | Nₜ | The total amount of data at time t |
| 90 | ŷ | The total amount of the predicted |
| | | values up to time t |
| 91 | E(ŷ) | The mathematical expectation |
| | | of vector ŷ |
| 92 | x_{IncTem} | The incinerator temperature at time t |
| 93 | X^{Dri} | Drift data |
| 94 | Z_{In}^{^{N}}^{In} | The output of the incremental layer |
| 95 | W^{N}^{_{FM}} ⁺ ^{N}^{_{En}} ⁺ ^{N}^{_{In}} ⁺ ^{N}^{_{OI}} | The weight between |
| | | G^{N}^{_{FM}} ⁺ ^{N}^{_{En}} ⁺ ^{N}^{_{In}} ⁺ ^{N}^{_{OI}} |
| | | and the predicted output |

This embodiment first builds a typical sample pool acquisition module based on k-means. Usually, historical data collected from complex industrial processes have the characteristics of high dimensionality, strong correlation, and high redundancy, resulting in modeling data and valuable information. There is an asymmetry problem between them. Therefore, this embodiment proposes a weighted k-means method to construct a typical sample pool (TSP). Theoretically, the same (or higher) modeling performance as the original data set can be obtained based on TSP.

Based on historical data D^{His}={xₙ, yₙ}ₙ ^{N}∈ [symbol] ^{N×(M+1)}, first randomly select I instances as the initial centroid, and then list all samples into category I based on the weighted Euclidean distance between the sample and the centroid:

$${C}_{i} = {C}_{i} ⋃ min {\{ {d}_{n} \}}_{n = 1}^{N} = {C}_{i} ⋃ min {\{ \sqrt{{[ {x}_{n} - {C}_{i} ]}^{2} {w}_{TS}^{T}} \}}_{n = 1}^{N} \qquad ( 1 )$$

Among them, Cᵢ represents the i-th class; w_{TS}^{T} represents the weight vector of the process variable, which is determined by the information value between the process variable and the DXN concentration, as follows:

$${w}_{T S}^{m} = H ( {x}_{m} ) - H ( {x}_{m} \vert y ) = {\sum }_{{x}_{n , m}} {\sum }_{{y}_{n}} p ( {x}_{n , m} , {y}_{n} ) {log}_{2} \frac{p ( {x}_{n , m} , {y}_{n} )}{p ( {x}_{n , m} ) p ( {y}_{n} )} ; \qquad ( 2 )$$

H(·) represents the information entropy of the random variable, xₘ is the m-th feature vector, y represents the DXN concentration, x_{n,m} represents the m-th eigenvalue of the n-th sample, p(x_{n,m}) and p(yₙ) represent the marginal probability distribution, and p(x_{n,m}, yₙ) is the joint probability distribution;

Update the centroid Cᵢ using inter-class samples:

$${C}_{i} = \frac{1}{{N}_{{C}_{i}}} {\sum }_{x ∈ {C}_{i}} {x}_{n} \qquad ( 3 )$$

Among them, N_{C}_{ᵢ} represents the number of samples in the i-th cluster;

The centroids are updated cyclically by using equations (1)-(3), and all centroids are obtained through preset conditions, which are expressed as:

$${\{ {C}_{i} \}}_{i}^{I} ⇐ \vert {R}_{i t e r} - {R}_{i t e r - 1} \vert \leq {δ}^{T S} \qquad ( 4 )$$

where δ^{TS} is the threshold of the evaluation index Rᵢₜₑᵣ, iter represents the number of iterations, and the calculation formula of the measurement index is:

$${R}_{i t e r} = {\sum }_{i = 1}^{I} {{\sum }_{n}^{{N}_{{C}_{i}}} [ {x}_{n} - {C}_{i} ]}^{2} {w}_{T S}^{T} \qquad ( 5 )$$

As can be seen from the above formula, it represents the sum of Euclidean distances between the cluster sample and the center of mass.

TSP is established by minimizing the clustering similarity, and the establishment formula is:

$${R}_{D B} = min ( \frac{1}{N} {\sum }_{n = 1}^{N} ( {S}_{i} + {S}_{j} ) / {M}_{i j} ) \qquad ( 6 )$$

Among them, R_{DB} is the clustering similarity measure index;

$${S}_{i} = {\{ ( 1 / {N}_{{C}_{i}} ) \sum {\vert ( {x}_{n} - {C}_{i} ) {w}_{T S}^{T} \vert}^{a} \}}^{1 / a} \qquad ( 7 )$$

$${M}_{ij} = {\{ \sum {\vert {C}_{i} - {C}_{j} \vert}^{b} \}}^{1 / b} \qquad ( 8 )$$

Where Sᵢ represents the sum of distances of the i-th category, and Mᵢⱼ represents the Minkowski metric criterion.

Secondly, this embodiment constructs a drift index calculation module based on PCA. For high-dimensional process variables, this embodiment uses a principal component analysis (PCA) model to calculate the drift index control limit used to determine whether the MSWI process changes.

First, the correlation coefficient matrix of TSP data D^{TSP}={xₙ, yₙ}ₙ^{N}^{_{TSP}}∈ [symbol] ^{N}^{_{TSP}}^{×(M+b)} is expressed as follows:

$$R \approx {\frac{1}{{N}_{T S P} - 1} [ {X}^{T S P} ]}^{T} {X}^{T S P} \qquad ( 9 )$$

where, N_{TSP} is the number of TSP data D^{TSP}; R is the correlation coefficient matrix of TSP data.

In order to obtain the potential variables that can represent the original high-dimensional process variables, that is, the principal components, this embodiment performs singular value decomposition (SVD) on R and calculates the eigenvalues:

$$R = {U}^{M \times M} {{Σ}^{M \times M} [ {V}^{M \times M} ]}^{T} \qquad ( 10 )$$

where, U^{M×M} and V^{M×M} represent orthogonal matrices V^{M×M}=[U^{M×M}]^{T}, and Σ^{M×M} is an M-dimensional pair angular matrix diag(σ₁, σ₂, . . . , σ_{M}) σₘ is the eigenvalue on the diagonal.

This embodiment feature cumulative contribution rate η and PCA contribution threshold δ^{PCA} for dimensionality reduction:

$$η = \frac{{\sum }_{{p}_{PCA} = 1}^{{P}_{PCA}} {σ}_{{p}_{PCA}}}{{\sum }_{m = 1}^{M} {σ}_{m}} \qquad ( 11 )$$

Among them, P_{PCA} is the number of selected principal components, and P_{PCA} is less than M. Accordingly, the drift control limit for monitoring whether the working conditions change is determined through P_{PCA} Further, rewrite equation (10) as:

$$\begin{matrix}R = {U}^{M \times M} {Σ}^{M \times M} {V}^{M \times M} \\ \approx {U}_{K e y}^{{P}_{P C A} \times {P}_{P C A}} {{Σ}_{K e y}^{{P}_{P C A} \times {P}_{P C A}} [ {U}_{K e y}^{{P}_{P C A} \times {P}_{P C A}} ]}^{T}\end{matrix} \qquad ( 12 )$$

where U_{Key}^{P}^{_{PCA}}^{×P}^{_{PCA}} is the load matrix;

According to the score matrix T and the load matrix U_{Key}^{P}^{_{PCA}}^{×P}^{_{PCA}} is expressed as:

$$\begin{matrix}{X}^{T S P} = {X}^{T S P} {{U}_{K e y}^{{P}_{P C A} \times {P}_{P C A}} [ {U}_{K e y}^{{P}_{P C A} \times {P}_{P C A}} ]}^{T} + {X}^{T S P} ( {I}^{{P}_{P C A} \times {P}_{P C A}} - {{U}_{K e y}^{{P}_{P C A} \times {P}_{P C A}} [ {U}_{K e y}^{{P}_{P C A} \times {P}_{P C A}} ]}^{T} ) \\ = {T [ {U}_{K e y}^{{P}_{P C A} \times {P}_{P C A}} ]}^{T} + {\tilde{T} [ {\tilde{U}}_{K e y}^{{P}_{P C A} \times {P}_{P C A}} ]}^{T} \\ = {\hat{X}}^{T S P} + {\tilde{X}}^{T S P}\end{matrix} \qquad ( 13 )$$

Among them, \hat{X}^{TSP} represents the projection of X^{TSP} on the principal component space, \hat{X}^{TSP} represents the projection of \tilde{X}^{TSP} on the residual space and satisfy the orthogonal relationship; In addition, and satisfies the orthogonal relationship, which is proved as follows:

$$\begin{matrix}{[ {\hat{X}}^{T S P} ]}^{T} {\tilde{X}}^{T S P} = {[ {X}^{T S P} {{U}_{K e y}^{{P}_{PCA} \times {P}_{P C A}} [ {U}_{K e y}^{{P}_{P C A} \times {P}_{PCA}} ]}^{T} ]}^{T} \cdot {X}^{T S P} ( {I}^{{P}_{PCA} \times {P}_{P C A}} - {{U}_{K e y}^{{P}_{PCA} \times {P}_{P C A}} [ {U}_{K e y}^{{P}_{P C A} \times {P}_{PCA}} ]}^{T} ) \\ = {{{U}_{K e y}^{{P}_{PCA} \times {P}_{PCA}} [ {U}_{K e y}^{{P}_{PCA} \times {P}_{P C A}} ]}^{T} [ {X}^{T S P} ]}^{T} {X}^{T S P} ( {I}^{{P}_{PCA} \times {P}_{P C A}} - {{U}_{K e y}^{{P}_{PCA} \times {P}_{P C A}} [ {U}_{K e y}^{{P}_{P C A} \times {P}_{PCA}} ]}^{T} ) \\ = {{{U}_{K e y}^{{P}_{PCA} \times {P}_{PCA}} [ {U}_{K e y}^{{P}_{PCA} \times {P}_{P C A}} ]}^{T} [ {X}^{T S P} ]}^{T} {X}^{T S P} {I}^{{P}_{PCA} \times {P}_{P C A}} - {{U}_{K e y}^{{P}_{P C A} \times {P}_{PCA}} [ {U}_{K e y}^{{P}_{P C A} \times {P}_{PCA}} ]}^{T} {[ {X}^{T S P} ]}^{T} {X}^{T S P} {{U}_{K e y}^{{P}_{PCA} \times {P}_{P C A}} [ {U}_{K e y}^{{P}_{P C A} \times {P}_{P C A}} ]}^{T}\end{matrix} \qquad ( 14 )$$

$$subject to {{U}_{K e y}^{{P}_{P C A} \times {P}_{PCA}} [ {U}_{K e y}^{{P}_{PCA} \times {P}_{P C A}} ]}^{T} = {I}^{{P}_{PCA} \times {P}_{P C A}}$$

$$= {[ {X}^{T S P} ]}^{T} {X}^{T S P} - {[ {X}^{T S P} ]}^{T} {X}^{T S P} = 0$$

Therefore, the advantage of using \hat{X}^{TSP} and \tilde{X}^{TSP} for process monitoring is that the two parts are independent of each other (that is, the statistical information does not interfere with each other).

This embodiment uses Hotelling's T² and SPE statistical indicators calculated based on TSP as the control limits for operating condition drift identification. The above two statistical indicators can be expressed as:

$${T}_{C L}^{2} = \frac{{P}_{P C A} ( {N}_{T S P} - 1 )}{( {N}_{T S P} - {P}_{P C A} )} {F}_{α} ( {P}_{P C A} , {N}_{T S P} - {P}_{P C A} ) \qquad ( 15 )$$

$$SP {E}_{CL} = {{Θ}_{1} ( \frac{{c}_{α} \sqrt{2 {Θ}_{2} {h}_{0}^{2}}}{{Θ}_{1}} + 1 + \frac{{Θ}_{2} {h}_{0} ( {h}_{0} - 1 )}{{Θ}_{1}^{2}} )}^{1 / {h}_{0}} \qquad ( 16 )$$

Among them, T_{CL}² is the control limit of Hotelling's T²; SPE_{CL} is the control limit of SPE; P_{PCA} is the number of selected principal components F_{α}(P_{PCA}, N_{TSP}−P_{PCA}) F_{α}(P_{PCA}, N_{TSP}−P_{PCA}) represents the F distribution with degrees of freedom of P_{PCA} and (N_{TSP}−P_{PCA}); c_{α}represents not normal deviation exceeding (1−α); Θ₁; Θ₂ and h₀ are calculated as follows:

$${h}_{0} = 1 - 2 {Θ}_{1} {Θ}_{3} / 3 {Θ}_{1}^{2} ; \qquad ( 17 )$$

$${Θ}_{i} = {\sum }_{m = {P}_{P C A} + 1}^{M} {( {σ}_{m} )}^{i} , i = 1 , 2 , 3 ; \qquad ( 18 )$$

Among them, Θ₁, Θ₂ and h₀, are all intermediate variables in SPE control limit calculation, and σₘ is the eigenvalue of singular value decomposition.

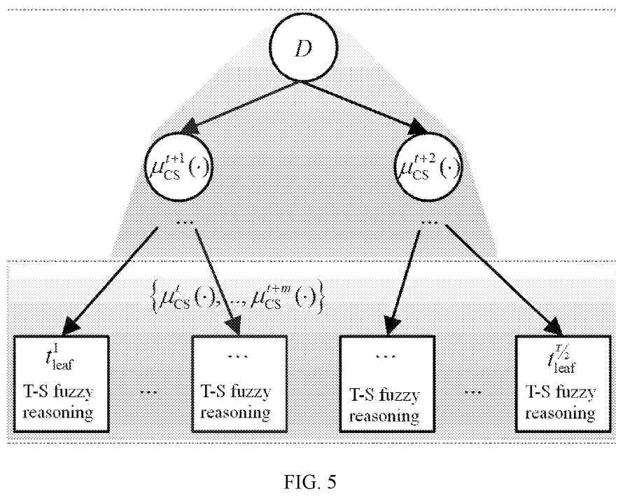

Thirdly, this embodiment builds an offline model building module based on FTBL. The FTBL offline modeling method consists of a feature mapping layer, an enhancement layer and an incremental layer (FIG. 5). Compared with traditional Decision Tree (DT), the basic units of each layer are replaced by FDT neurons. The structure of FDT is shown in FIG. 5. FDT is a type of binary tree. Its structure includes non-leaf nodes and leaf nodes. The antecedent part of Takagi-Sugeno (TS) fuzzy reasoning is used for feature selection and the consequent part of TS fuzzy reasoning is used for TS fuzzy inference system.

### 1) Feature Mapping Layer

For the given TSP data D^{TSP}={xₙ, yₙ}ₙ ^{N}^{_{TSP}}∈ [symbol] ^{N}^{_{TSP}}^{x(M+1)}, randomly select a feature value^{x} in D^{TSP} to define node splitting function μ_{CS}^{t}(x), where:

$${μ}_{C S}^{t} ( x ) = r a n d ( n , m ) , n ∈ ( 1 , {N}_{TSP} ) and m ∈ ( 1 , M ) \qquad ( 19 )$$

Wherein μ_{CS}(x) is a symbolic function, rand(·) is a random number generation function, n and m do not take the maximum and minimum values.

This embodiment can obtain T/2−1 non-leaf nodes ({μ_{CS}^{t}(x)}ₜ₌₁^{T/2-1}) and T/2 leaf nodes of FDT constructed by equation (20). Since the paths from the root node to the leaf nodes are different, the input data D^{Leaf}={xₙ^{Leaf}, yₙ}ₙ ^{N}^{_{TSP}}^{^{Leaf}}∈ [symbol] ^{N}^{_{TSP}}^{^{Leaf}}^{x(M}^{^{Leaf}}⁺¹⁾, of each leaf node is different. Therefore, the D^{Leaf}-based TS fuzzy inference process is as follows

Determine K fuzzy rules for TS fuzzy reasoning, and the k-th rule can be expressed as:

$${R}_{k} : if {x}_{1}^{{t}_{leaf}} is {φ}_{1}^{k} ( {x}_{1}^{{t}_{leaf}} ) and … and {x}_{{M}^{{t}_{leaf}}}^{{t}_{leaf}} is {μ}_{{M}^{{t}_{leaf}}}^{k} ( {x}_{{M}^{{t}_{leaf}}}^{{t}_{leaf}} ) \qquad ( 20 )$$

$$then {y}^{k} = {g}^{k} ( {x}_{1} , … , {x}_{{M}^{{t}_{leaf}}} )$$

$$\text{Wherein:}$$

$${φ}_{m}^{k} ( {x}_{m}^{{t}_{leaf}} ) = exp [ - {( {x}_{m}^{{t}_{leaf}} - {c}_{k , m} )}^{2} / {σ}_{k , m}^{2} ] \qquad ( 21 )$$

Among them, Rₖ is the k-th fuzzy rule, c_{k,m} and σ_{k,m} respectively represent the center and width of the Gaussian function φₘ^{k}(·), t_{leaf} represents the t_{leaf}-th leaf node; φₘ^{k}(·) is the Gaussian function.

According to the above K fuzzy rules, the result of FDT is described as:

$${\hat{y}}_{i} = f ( {x}_{i} ) = {\sum }_{k = 1}^{K} {\overline{o}}^{k} {g}^{k} ( {x}_{i} ) \qquad ( 22 )$$

$$\text{Wherein:}$$

$${\overline{o}}^{k} = {\prod }_{m = 1}^{{M}^{Leaf}} {φ}_{m}^{k} ( {x}_{m}^{{t}_{leaf}} ) / {\sum }_{i = 1}^{K} ( {\prod }_{m = 1}^{{M}^{Leaf}} {φ}_{m}^{k} ( {x}_{m}^{{t}_{leaf}} ) ) \qquad ( 23 )$$

$${g}^{k} ( {x}_{i}^{Leaf} ) = {x}_{i}^{Leaf} {ω}_{TS}^{k} \qquad ( 24 )$$

Among them, f(·) is the FDT model, ō^{k} and g^{k}(·) represents the antecedent and consequent parts of TS fuzzy reasoning, and ω_{TS}^{k} represents the weight of the features of the consequent part.

This embodiment apply the gradient descent method to update the parameters during the training process of the FDT model f(·). The parameters include the center cₖ, the width σₖ and the weight ω_{TS}^{k}. The output of the feature mapping layer is expressed as follows:

$${Z}_{FM}^{{N}_{FM}} = [ {z}_{FM}^{1} , {z}_{FM}^{2} , … , {z}_{FM}^{{N}_{FM}} ] ∈ {ℝ}^{{N}_{TSP} \times {N}_{FM}} \qquad ( 25 )$$

$$\text{where:}$$

$${z}_{FM}^{{n}_{FM}} = {f}_{{n}_{FM}}^{FM} ( {X}^{TSP} ) ∈ {ℝ}^{{N}_{TSP} \times 1} \qquad ( 26 )$$

where z_{FM}ⁿ^{_{FM}} is the output of the n_{FM}-th FDT model through the input X^{TSP}.

### 2) Enhancement Layer

The enhancement layer takes Z_{FM}^{N}^{_{FM}} as input, and the output of the enhancement layer is expressed as:

$${Z}_{En}^{{N}_{En}} = [ {z}_{En}^{1} , {z}_{En}^{2} , … , {z}_{En}^{{N}_{En}} ] ∈ {ℝ}^{{N}_{TSP} \times {N}_{En}} \qquad ( 27 )$$

The output of the feature mapping layer and the enhancement layer is G^{N}^{_{FM}}^{+N}^{_{En}}=[Z_{FM}^{N}^{_{FM}}|Z_{En}^{N}^{_{En}}]. Use the ridge regression learning algorithm to calculate the weight W^{N}^{_{FM}}^{+N}^{_{En}} between G^{N}^{_{FM}}^{+N}^{_{En}} and the predicted output:

$$\begin{matrix}{W}^{{N}_{FM} + {N}_{En}} = {( λ I + {{G}^{{N}_{FM} + {N}_{En}} ( {G}^{{N}_{FM} + {N}_{En}} )}^{T} )}^{- 1} {( {G}^{{N}_{FM} + {N}_{En}} )}^{T} {y}^{TSP} \\ = {( {G}^{{N}_{FM} + {N}_{En}} )}^{*} {y}^{TSP}\end{matrix} \qquad ( 28 )$$

Among them, (G^{N}^{_{FM}}^{+N}^{_{En}})* is the pseudo-inverse matrix, λ is the regularization coefficient, and I is the identity matrix.

### 3) Incremental Layer

In order to obtain good enough performance, the FDT model is further added to the incremental layer and the pseudo-inverse matrix is dynamically updated.

Taking Z_{En}^{N}^{_{En}} as input and G^{N}^{_{FM}}^{+N}^{_{En}}⁺¹=[Z_{FM}^{N}^{_{FM}}|Z_{En}^{N}^{_{En}}|z_{In}¹]as output; the pseudo-inverse matrix update process of the incremental process is as follows:

$${[ {G}^{{N}_{FM} + {N}_{En} + 1} ]}^{*} = [ \begin{matrix}{[ {G}^{{N}_{FM} + {N}_{En} + 1} ]}^{*} - {DB}^{T} \\ {B}^{T}\end{matrix} ] \qquad ( 29 )$$

$$\text{Wherein:}$$

$$\{ \begin{matrix}D = {[ {G}^{{N}_{FM} + {N}_{En} + 1} ]}^{*} {H}_{k + 1} \\ {B}^{T} = \{ \begin{matrix}{[ C ]}^{*} , & if C \neq 0 \\ {[ 1 + {D}^{T} D ]}^{- 1} {{D}^{T} [ {G}^{{N}_{FM} + {N}_{En} + 1} ]}^{*} , & if C = 0\end{matrix} \\ C = {H}_{k + 1} - {G}^{{N}_{FM} + {N}_{En} + 1} D\end{matrix} \qquad ( 30 )$$

Among them, Hₖ₊₁ B^{T} and C are all intermediate variables in the pseudo-inverse matrix update process.

The new weight matrix W^{N}^{_{FM}}^{+N}^{_{En}}⁺¹ is expressed as:

$${W}^{{N}_{FM} + {N}_{En} + 1} = [ \begin{matrix}{W}^{{N}_{FM} + {N}_{En}} - {DB}^{T} {y}^{TSP} \\ {B}^{T} {y}^{TSP}\end{matrix} ] \qquad ( 31 )$$

The prediction calculation process of the offline model FTBL is as follows:

$$\hat{y} = {G}^{{N}_{FM} + {N}_{En} + 1} {W}^{{N}_{FM} + {N}_{En} + 1} \qquad ( 32 )$$

This embodiment also constructs an online drift identification and soft measurement module, which performs real-time monitoring of the MSWI process based on the PCA model and drift indicator control limits established above, and performs corresponding soft measurement output based on the drift identification results. This embodiment uses a fixed window size to acquire process data, and starts drift identification and soft measurement when the data fills the window.

Calculate the drift value within the new window based on:

$${T}_{{O}_{1}}^{2} = {x}_{{O}_{1}}^{T} {U}_{{N}_{TSP} + 1}^{{P}_{PCA} \times {P}_{PCA}} {\sum }_{{N}_{TSP} + 1}^{- 1} {( {U}_{{N}_{TSP} + 1}^{{P}_{PCA} \times {P}_{PCA}} )}^{T} {x}_{{O}_{1}} \qquad ( 33 )$$

$${SPE}_{{O}_{1}} = {x}_{{N}_{TSP} + 1}^{T} ( I - {{U}_{{N}_{TSP} + 1}^{{P}_{PCA} \times {P}_{PCA}} ( {U}_{{N}_{TSP} + 1}^{{P}_{PCA} \times {P}_{PCA}} )}^{T} ) {x}_{{N}_{TSP} + 1} \qquad ( 34 )$$

Among them, Tₒ_{₁}² and SPEₒ_{₁} are the statistical indicators of the (N_{TSP}+1)-th process data x_{O}_{₁}, U_{N}_{_{TSP}}₊₁^{P}^{_{PCA}}^{×P}^{_{PCA}} represent the new load matrix,

$${\sum }_{{N}_{TSP} + 1}^{- 1}$$

represent the new diagonal matrix diag(σ₁, σ₂, . . . , σ_{P}_{_{PCA}}).

Determine whether the sample is a drift sample or a normal sample through a judgment formula; the judgment formula is:

$$\{ \begin{matrix}drift & if {T}_{{O}_{1}}^{2} > {T}_{CL}^{2} and {SPE}_{{O}_{1}} > {SPE}_{CL} \\ normal & etc .\end{matrix} \qquad ( 35 )$$

For normal samples, the offline model of FTBL is reused for soft measurement of DXN concentration, which can be expressed as:

$$\begin{matrix}{\hat{y}}_{{O}_{t}} = {G}^{k + {N}_{In}} {W}^{k + {N}_{In}} \\ = [ {Z}_{FM}^{{N}_{FM}} \vert {Z}_{En}^{{N}_{En}} \vert {Z}_{In}^{{N}_{In}} ] {W}^{k + {N}_{In}} \\ = [ {f}_{1}^{FM} ( {x}_{{O}_{t}} ) , … , {f}_{{N}_{FM}}^{FM} ( {x}_{{O}_{t}} ) \vert {f}_{1}^{En} ( {x}_{{O}_{t}} ) , … , \\ {f}_{{N}_{En}}^{En} ( {x}_{{O}_{t}} ) \vert {f}_{1}^{In} ( {x}_{{O}_{t}} ) , … , {f}_{{N}_{In}}^{In} ( {x}_{{O}_{t}} ) ] {W}^{k + {N}_{In}}\end{matrix} \qquad ( 36 )$$

Considering that the true value detection of DXN emissions in actual projects takes a long time and it is difficult to obtain the true value data of DXN in real time, in addition to prompting the need for detection, the following soft measurement value calculation method is given here:

$${\hat{y}}_{{O}_{t}} = {G}^{k + {N}_{In}} {W}^{k + {N}_{In}} + {ε}_{Offset} \qquad ( 37 )$$

Among them, ε_{Offset} is the offset value of offline FTBL prediction output is as follows:

$${ε}_{Offset} = \{ \begin{matrix}+ \frac{1}{{N}_{t}} \sum ( {\hat{y}}_{t} - E ( \hat{y} ) ) , if {x}_{IncTem} < E ( {x}_{IncTem} ) \\ - \frac{1}{{N}_{t}} \sum ( {\hat{y}}_{t} - E ( \hat{y} ) ) , if {x}_{IncTem} \geq E ( {x}_{IncTem} )\end{matrix} \qquad ( 38 )$$

Among them, Nₜ represents the total amount of data arriving at time t, ŷ represents all the predicted values arriving at time t, E(ŷ) represents the mathematical expectation of the vector ŷ, and X_{IncTem} represents the incinerator temperature at time t.

In addition, when a true value exists, the following method is used to update FTBL. Finally, this embodiment also constructs an online dynamic update module for the FTBL model. For drift data, this embodiment adds an online incremental layer to quickly learn the characteristics of the drift data. In the online incremental process, the offline FTBL is described as an independent model, which enhances the online measurement accuracy of the model by extending the concept of the original model width. At this time, the input data includes TSP data (X_{TSP}), drift data (X_{Dri}) and the output of the incremental layer. The predicted value of the new FTBL model is recorded as:

$${\hat{y}}_{{O}_{t}} = {G}^{{N}_{FM} + {N}_{En} + {N}_{In} + {N}_{OI}} {W}^{{N}_{FM} + {N}_{En} + {N}_{In} + {N}_{OI}} \qquad ( 39 )$$

Among them, W^{N}^{_{FM}}^{+N}^{_{En}}^{+N}^{_{In}}^{+N}^{_{OI}} represents the weight matrix, and G^{N}^{_{FM}}^{+N}^{_{En}}^{+N}^{_{In}}^{+N}^{_{OI}} is the FDT output matrix of N_{FM}+N_{En}+N_{In}+N_{OI}, and its dynamic update process is consistent with equations (29) to (31).

After completing online learning and prediction, the TSP and SPE_{CL} control limits need to be updated to adapt to the online measurement in the next window.

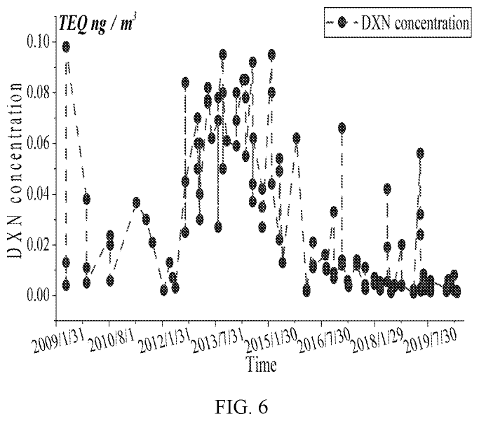

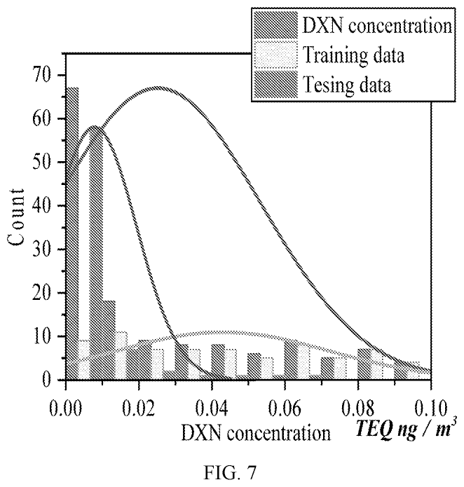

As shown in FIGS. 6 and 7, this embodiment uses the DXN data of a large MSWI power plant in Beijing from 2009 to 2020. The left side of FIG. 6 and FIG. 7 shows the actual concentration value of DXN.

As can be seen from FIGS. 6 and 7, the DXN concentration of the MSWI power plant has not exceeded 0.1 TEQ ng/m³ since it was put into operation, meeting the requirements of the pollutant control emission standards (GB18485-2014). Considering that it is difficult to obtain the true value of DXN, an offline model can only be established for soft measurement driven by historical data. Therefore, the historical data takes the true value samples from 2009 to 2016, and the test data takes the true value samples from 2016 to 2020. The true value of DXN concentration is the average emission concentration of the MSWI process within 2 hours. At the same time, in discrete control systems (DCS), process variables such as temperature, pressure, and flow are generated within seconds, and it is necessary to average the process data within the sampling time to obtain samples corresponding to the true DXN values. The division of DXN data in this embodiment is shown in Table 2.

TABLE 2
DXN data division

| | data | training set | test set |
|---|---|---|---|
| sample | 141 | 71 | 70 |
| feature | 116 | 116 | 116 |
| time | 2009 s~2020 s | 2009 s~2016 s | 2016 s~2020 s |

It can be seen from FIG. 7 that there is a significant difference in the distribution of historical data and test data, which will make it difficult to accurately detect DXN emission concentration based on the classic modeling method.

Using classic and popular methods such as Random Forest (RF), Backpropagation Neural Network (BPNN), Deep Forest Regression (DFR), Support Vector Regression (SVR), Fuzzy Neural Network (FNN) and Breadth Learning System (BLS) to compare with the proposed FTBL method.

The indicators used are root mean square error (RMSE) and explained variance (EV), which are calculated as follows:

$$RMSE = \sqrt{\frac{1}{N} {\sum }_{n = 1}^{N} {( \hat{y} - {y}_{n} )}^{2}} \qquad ( 40 )$$

$$EV = 1 - \frac{{\sum }_{n = 1}^{N} {( ( \hat{y} - {y}_{n} ) - E ( \hat{y} - y ) )}^{2}}{{\sum }_{n = 1}^{N} {( {y}_{n} - E ( y ) )}^{2}} \qquad ( 41 )$$

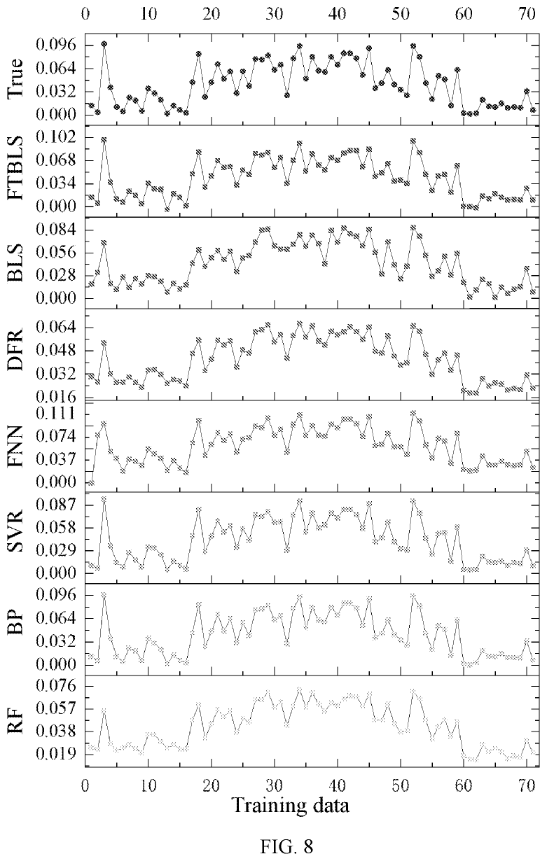

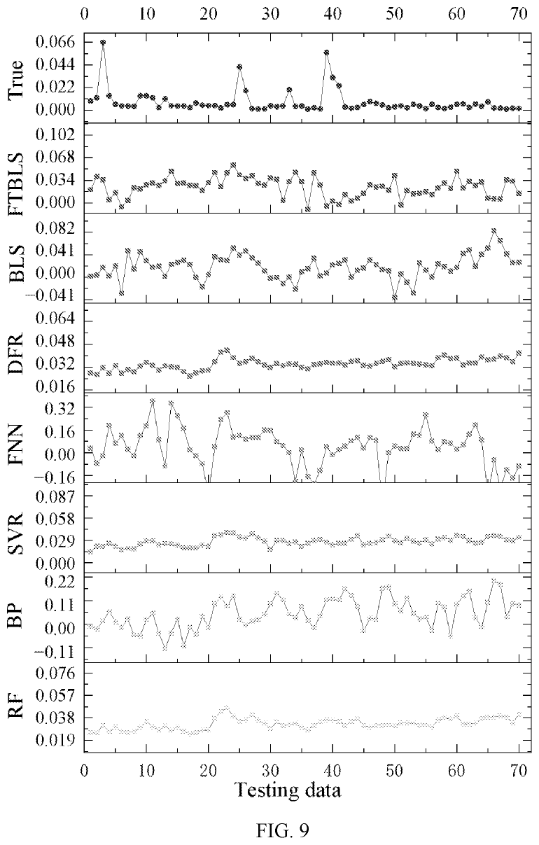

The experimental data and fitting curves are shown in Table 3 and FIGS. 8 and 9.

TABLE 3
Comparison results of different methods

| | training set | | test set | |
|---|---|---|---|---|
| | (2009 s~2016 s) | | (2016 s~2020 s) | |
| method | RMSE | EV | RMSE | EV |
| RF | 1.3248E−02 | 7.9134E−01 | 2.7447E−02 | −1.9795E−01 |
| BP | 9.2128E−04 | 9.9899E−01 | 5.7262E−02 | −1.6648E+01 |
| SVR | 6.7068E−03 | 7.7486E−01 | 2.0233E−02 | −6.8958E+00 |
| FNN | 2.2107E−03 | 9.9419E−01 | 1.1513E−01 | −8.4589E+01 |
| DFR | 1.5410E−02 | 7.1763E−01 | 2.8644E−02 | −1.4681E−01 |
| BLS | 1.0074E−02 | 8.7950E−01 | 2.6993E−02 | −2.4269E+00 |
| Offline | 7.1257E−03 | 9.3979E−01 | 2.3639E−02 | −1.9953E+00 |
| FTBL | | | | |

It can be seen from Table 3 and FIG. 8 and FIG. 9 that the modeling performance of the above methods is good enough, specifically:
(1) In the training data, the RMSE and EV of BPNN, FNN and FTBL are better than those of RF, DFR, SVR and BLS, which shows that BPNN, FNN and FTBL have better fitting performance for the training data;
(2) In the test data, due to differences in data distribution, there are significant differences in prediction performance, making it difficult for all methods to effectively fit the distribution of the test data;
(3) SVR has the lowest RMSE (2.0233E-02) and RF has the lowest EV (−1.9795E-01), while the prediction curves of the SVR, RF and DFR methods approach a straight line, which means the response ability of RF, SVR and DFR to test data is poor; in addition, other methods such as BP, FNN, BLS and FTBL are more sensitive to online data. This shows that the prediction trends of these methods fluctuate with changes in the online process. The FTBL method proposed in this embodiment has high advantages in both modeling accuracy and model sensitivity.

The above experimental results show that the proposed offline modeling method has higher modeling accuracy and sensitivity than RF and DFR methods; in addition, compared with BP, FNN, SVR and BLS methods, FTBL has the same modeling accuracy, higher sensitivity and stable sensitivity.

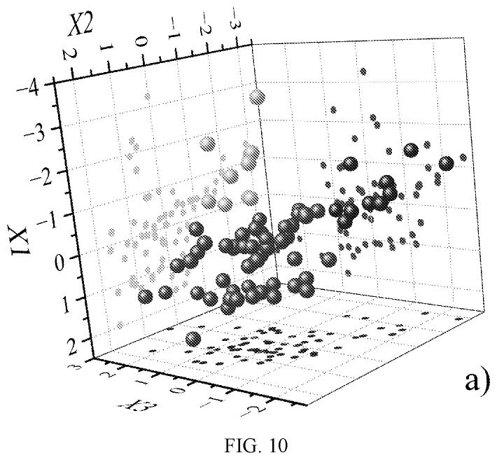

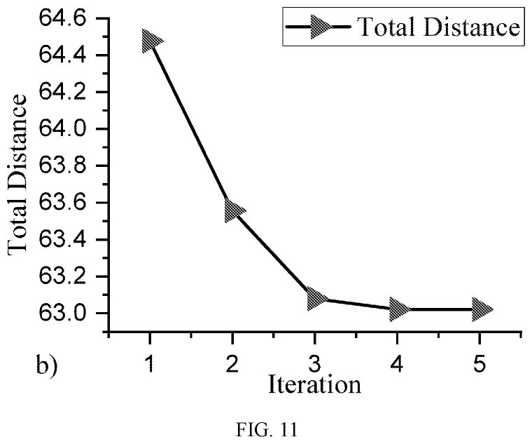

This embodiment uses weighted k-means to cluster training data and uses CSM indicators to construct TSP data DTSP. The clustering results take the first three dimensions x₁, x₂, and x₃ of historical data as an example. The results are shown in FIGS. 10 and 11.

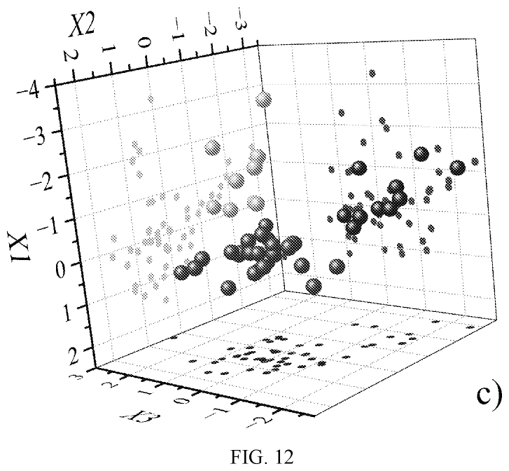

FIGS. 10 and 11 demonstrate the effectiveness of weighted k-means clustering in three-dimensional space, with clear spatial distances between clusters. During the iterative process, the total distance Rᵢₜₑᵣ between clusters gradually decreases until convergence (FIG. 11). Then, redundant samples are removed based on CSM indicators. The results are shown in FIG. 12 and Table 4. Comparing the results in FIG. 10 and FIG. 12, we can see that the samples within a class are closer to the periphery of the centroid, and the distance between classes has been expanded. In addition, judging from the statistical results of the CSM index, the clustering similarity of typical samples is lower.

TABLE 4
CSM of typical samples

| | CSM index | sample |
|---|---|---|
| old | 3.0452E+01 | 71 |
| new | 2.7603E+01 | 64 |

In this embodiment, the contribution threshold used to determine the principal components is δ^{PCA}=0.9, and 19 principal components are selected for statistical analysis. According to equations (15), (16) and 19 principal components, the two control limits of historical data and SPE_{CL} are further obtained, which are 13.2270 and 19.3475 respectively, with a confidence level of 95%.

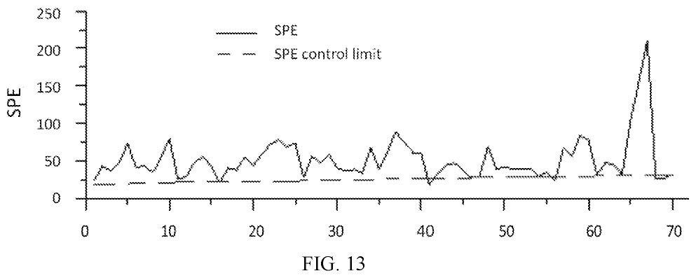

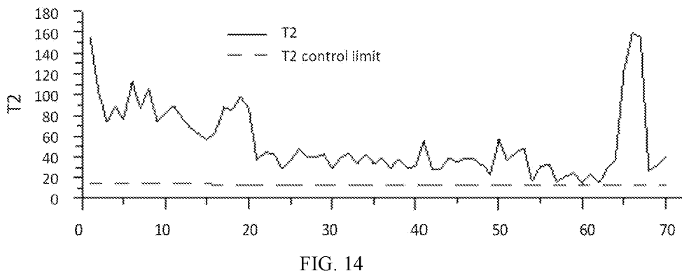

The moving window size of fixed online monitoring is 5. Based on the offline model established in the previous section, the online drift identification and soft measurement results are shown in FIG. 13, FIG. 14 and Table 5.

TABLE 5
Results of the online measurement process

| | | | Online | | Update Time |
|---|---|---|---|---|---|
| Window | Size | Drift | incremental layer | RMSE | (s) |
| W1 | 5 | 5 | 5 | 2.8219E−03 | 7.0182E−01 |
| W2 | 5 | 5 | 5 | 2.2935E−03 | 4.5206E−01 |
| W3 | 5 | 5 | 5 | 2.8391E−03 | 6.0348E−01 |
| W4 | 5 | 5 | 5 | 1.5100E−03 | 6.0865E−01 |
| W5 | 5 | 5 | 5 | 3.2899E−03 | 6.0067E−01 |
| W6 | 5 | 5 | 5 | 1.9852E−03 | 7.1308E−01 |
| W7 | 5 | 5 | 5 | 2.2766E−03 | 7.0608E−01 |
| W8 | 5 | 5 | 5 | 1.9585E−03 | 6.9887E−01 |
| W9 | 5 | 5 | 5 | 1.6349E−03 | 5.9082E−01 |
| W10 | 5 | 5 | 5 | 4.0845E−03 | 5.8389E−01 |
| W11 | 5 | 5 | 5 | 4.2041E−03 | 7.2849E−01 |
| W12 | 5 | 5 | 5 | 1.6638E−03 | 6.2889E−01 |
| W13 | 5 | 5 | 5 | 1.6363E−03 | 5.4703E−01 |
| W14 | 5 | 5 | 5 | 1.8561E−03 | 5.8797E−01 |

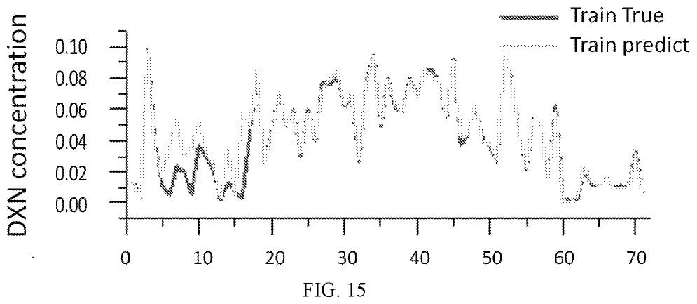

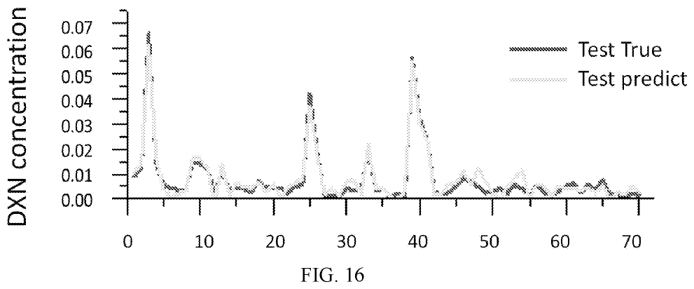

Experimental results show that there are significant differences between the process data in the online phase and the historical data. According to the control limits of data statistical indicators, it can be seen that all process data are drift samples. Setting the number of FDTs in the online incremental update layer to 5 which effectively controls the time cost of soft measurement. The results in Table 5 show that the FTBL online update process has high fitting accuracy and short update time. The corresponding experimental results are shown in FIGS. 15 and 16.

As shown in Table 6, the RMSE and EV of the historical data are 9.9065E-03 and 8.9321E-01 respectively, and the RMSE and EV of the test data are 2.1595E-02 and 9.5089E-01 respectively. Consider the following aspects: 1) The offline FTBL model uses TSP. Although its modeling accuracy is slightly reduced, the modeling time cost and the computational cost of the online update process are reduced; 2) Compared with the offline modeling part, the test and monitoring phase achieves better fitting of data and higher modeling accuracy. The results show that the offline modeling and online measurement strategies proposed in this embodiment are sufficient.

TABLE 6
Statistics of prediction indicators in offline and online stages

| stage | RMSE | EV |
|---|---|---|
| Train | 9.9065E−03 | 8.9321E−01 |
| Test | 2.1595E−02 | 9.5089E−01 |

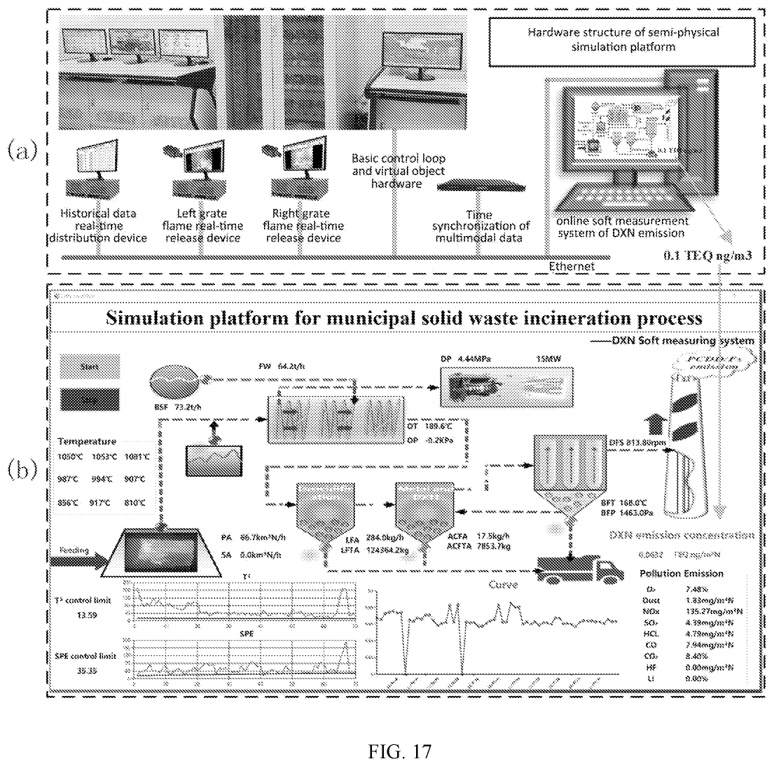

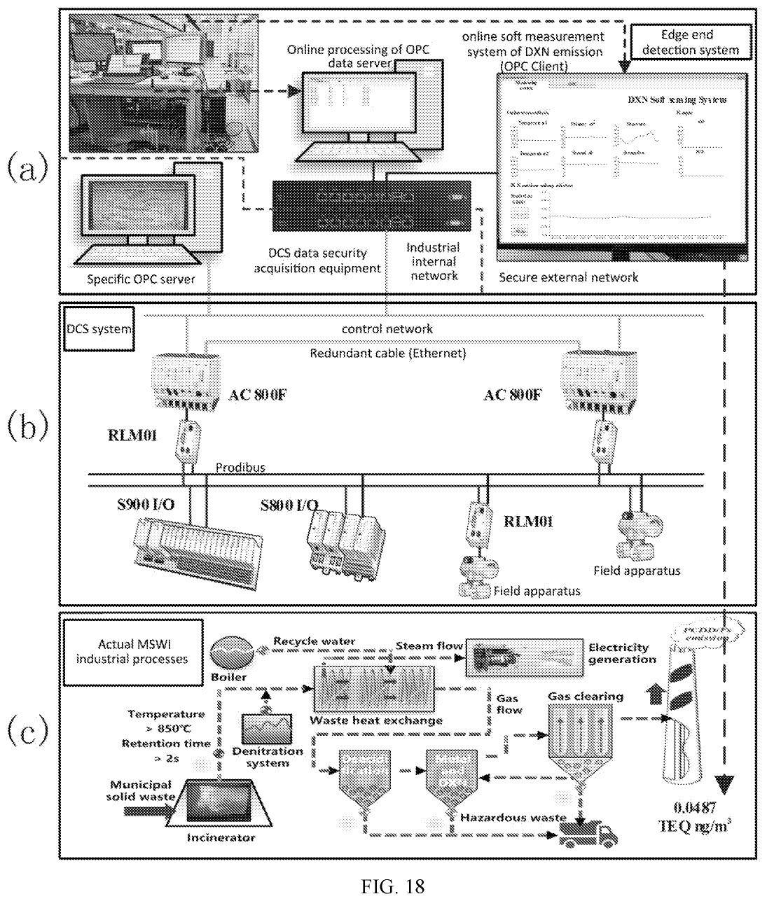

In view of the working condition of drift, the proposed strategy is implemented here based on actual engineering applications. In this embodiment, a DXN concentration emission online detection system was developed based on the C# programming language, and experimental tests were conducted on the laboratory semi-physical simulation platform and MSWI industrial site. The corresponding hardware structure and software interface are shown in FIGS. 17 and 18.

In this embodiment, after starting the operation through the “Start” button, the software interface displays the online predicted DXN emission concentration in real time. Above the software interface is the hardware structure of the semi-physical simulation platform. The results of testing the soft measurement system on a semi-physical simulation platform verify the effectiveness of the software system, indicating that the method proposed in this embodiment can be applied in engineering and provide strong support for actual projects.

The beneficial effects of the present invention are as follows:

Aiming at the soft measurement problem of DXN emission concentration in the MSWI process, this embodiment proposes a soft measurement strategy based on fuzzy tree width learning. The main contributions are reflected in: proposing a new FTBL algorithm to build an offline DXN emission model, and proposing online operating condition of drift. The corresponding soft sensing strategy was identified and an online dynamic update method of the FTBL model was proposed. After verifying the effectiveness of the proposed strategy on the laboratory simulation experiment platform based on actual process data, the proposed method was verified in the actual industrial process.

Each embodiment in this specification is described in a progressive manner. Each embodiment focuses on its differences from other embodiments. The same and similar parts between the various embodiments can be referred to each other.

In this embodiment, specific examples are used to illustrate the principles and implementation methods of the present invention. The description of the above embodiments is only used to help understand the method and the core idea of the present invention; at the same time, for those of ordinary skill in the art, based on the idea of the present invention will have changes in the specific implementation and application scope. In summary, the contents of this description should not be construed as limitations of the present invention.

1. An online soft measurement method for dioxin emission concentration in a municipal solid waste incineration (MSWI) process, comprising:
based on a K-means weighting algorithm, determining process data of a typical sample pool based on a historical process data set of MSWI;
performing principal component analysis based on the process data of the typical sample pool to obtain a drift indicator control limit, wherein the drift indicator control limit reflects whether the MSWI process has changed;
constructing an offline model based on Fuzzy Tree-Based Learning (FTBL), and inputting the process data of the typical sample pool and historical dioxin (DXN) true value data of MSWI into the offline model for prediction calculation to obtain an offline calculation result; wherein the offline model comprises a feature mapping layer, an enhancement layer and an incremental layer;
performing principal component analysis based on online data, and determining whether the online data is drift data or normal data based on the drift indicator control limit; when the online data is the normal data, jumping to the step “constructing an offline model based on FTBL, and inputting the process data of the typical sample pool and historical DXN true value data of MSWI into the offline model for prediction calculation to obtain an offline calculation result”; when the online data is the drift data, constructing an online model based on FTBL, and inputting the process data of the typical sample pool, the drift data, and output data of the incremental layer of the offline model into the online model for prediction calculation to obtain an online calculation result; wherein the online model comprises an online incremental layer; and
determining a DXN emission concentration prediction value based on the offline calculation result and the online calculation result.

2. The online soft measurement method for dioxin emission concentration in the MSWI process according to claim 1, wherein the step of based on the K-means weighting algorithm, determining the process data of the typical sample pool based on the historical process data set of MSWI comprises:
getting the historical process data set X^{His} of MSWI;
obtaining historical data D^{His}={xₙ,yₙ}ₙ^{N}∈ [symbol] ^{N×(M+1)} according to the historical process data set X^{His};
wherein xₙ represent an n-th sample, yₙ represent a predict value of the n-th sample, N represent a number of sample in the historical process data set of MSWI, and M represent a feature number in the historical process data set of MSWI;
randomly selecting/instance as an initial centroid {Cᵢ}ᵢ₌₁^{I}.
listing all samples into class/according to a weighted Euclidean distance between the sample and the centroid:
$${C}_{i} = {C}_{i} ⋃ min {\{ {d}_{n} \}}_{n = 1}^{N} = {C}_{i} ⋃ min {\{ \sqrt{{[ {x}_{n} - {C}_{i} ]}^{2} {w}_{TS}^{T}} \}}_{n = 1}^{N}$$
wherein, Cᵢ represents an i-th class; w_{TS}^{T} represents a weight vector of a process variable, wherein
$${w}_{TS}^{m} = H ( {x}_{m} ) - H ( {x}_{m} \vert y ) = {\sum }_{{x}_{n , m}} {\sum }_{{y}_{n}} p ( {x}_{n , m} , {y}_{n} ) {log}_{2} \frac{p ( {x}_{n , m} , {y}_{n} )}{p ( {x}_{n , m} ) p ( {y}_{n} )} ;$$
H(·) represents information entropy of a random variable, xₘ is an m-th feature vector, y represents a DXN concentration, x_{n,m} represents an m-th eigenvalue of the n-th sample, p(x_{n,m}) and p(yₙ) represent a marginal probability distribution, and p(x_{n,m}, yₙ) is a joint probability distribution;
updating the centroid Cᵢ using inter-class samples:
$${C}_{j} = \frac{1}{{N}_{{C}_{i}}} {\sum }_{x ∈ {C}_{i}} {X}_{n}$$
wherein, N_{C}_{ᵢ} represents a number of samples in an i-th cluster;
cyclically updating the centroids, and obtaining all centroids through preset conditions, wherein the preset conditions are expressed as:
$${\{ {C}_{i} \}}_{i}^{I} ⇐ \vert {R}_{i t e r} - {R}_{i t e r - 1} \vert \leq {δ}^{TS}$$
where δ^{TS} is a threshold of an evaluation index Rᵢₜₑᵣ, iter represents a number of iterations, and a calculation formula of a measurement index is:
$${R}_{i t e r} = {\sum }_{i = 1}^{I} {{\sum }_{n}^{{N}_{{C}_{i}}} [ {x}_{n} - {C}_{i} ]}^{2} {W}_{TS}^{T}$$
establishing typical sample pool (TSP) by minimizing a clustering similarity, wherein an establishment formula is:
$${R}_{D B} = min ( \frac{1}{N} {\sum }_{n = 1}^{N} ( {S}_{i} + {S}_{j} ) / {M}_{i j} )$$
wherein, R_{DB} is a clustering similarity measure index;
$${S}_{i} = {\{ ( 1 / {N}_{{C}_{i}} ) \sum {\vert ( {x}_{n} - {C}_{i} ) {w}_{TS}^{T} \vert}^{a} \}}^{1 / a} {M}_{ij} = {\{ \sum {\vert {C}_{i} - {C}_{j} \vert}^{b} \}}^{1 / b}$$
wherein Sᵢ represents a sum of distances of an i-th category, and Mᵢⱼ represents a Minkowski metric criterion.

3. The online soft measurement method for dioxin emission concentration in the MSWI process according to claim 2, wherein the step of performing principal component analysis based on the process data of the typical sample pool to obtain the drift indicator control limi comprises:
expressing a correlation coefficient matrix of TSP data
$${D}^{TSP} = {\{ {x}_{n} , {y}_{n} \}}_{n}^{{N}_{TSP}} ∈ {ℝ}^{{N}_{TSP} \times ( M + 1 )} as R \approx {\frac{1}{{N}_{TSP} - 1} [ {X}^{TSP} ]}^{T} {X}^{TSP} ,$$
wherein, N_{TSP} is a number of TSP data D^{TSP}; R is a correlation coefficient matrix of TSP data;
performing singular value decomposition on R and calculating eigenvalues; wherein a calculation formula is R=U^{M×M}Σ^{M×M}[V^{M×M}]^{T}; wherein, U^{M×M} and V^{M×M} represent orthogonal matrices, and Σ^{M×M} is an M-dimensional pair angular matrix;
using feature cumulative contribution rate η and principal component analysis (PCA) contribution threshold δ^{PCA} for dimensionality reduction:
$$η = \frac{{\sum }_{{p}_{PCA} = 1}^{{P}_{PCA}} {σ}_{{p}_{PCA}}}{{\sum }_{m = 1}^{M} {σ}_{m}} ;$$
wherein, P_{PCA} is a number of selected principal components, and P_{PCA} is less than M; rewriting a calculation formula as:
$$R = {U}^{M \times M} {\sum }^{M \times M} {V}^{M \times M} \approx {U}_{Key}^{{P}_{PCA} \times {P}_{PCA}} {{\sum }_{Key}^{{P}_{PCA} \times {P}_{PCA}} [ {U}_{Key}^{{P}_{PCA} \times {P}_{PCA}} ]}^{T}$$
wherein U_{Key}^{P}^{_{PCA}}^{×P}^{_{PCA}} is a load matrix;
according to a score matrix T and a load matrix U_{Key}^{P}^{_{PCA}}^{×P}^{_{PCA}}, expressing X^{TPS} as:
$$\begin{matrix}{X}^{TSP} = {X}^{TSP} {{U}_{Key}^{{P}_{PCA} \times {P}_{PCA}} [ {U}_{Key}^{{P}_{PCA} \times {P}_{PCA}} ]}^{T} + \\ {X}^{TSP} ( {I}^{{P}_{PCA} \times {P}_{PCA}} - {{U}_{Key}^{{P}_{PCA} \times {P}_{PCA}} [ {U}_{Key}^{{P}_{PCA} \times {P}_{PCA}} ]}^{T} ) \\ = {T [ {U}_{Key}^{{P}_{PCA} \times {P}_{PCA}} ]}^{T} + {\tilde{T} [ {\tilde{U}}_{Key}^{{P}_{PCA} \times {P}_{PCA}} ]}^{T} \\ = {\hat{X}}^{TSP} + {\tilde{X}}^{TSP}\end{matrix}$$
wherein, \hat{X}^{TSP} represents a projection of X^{TSP} on a principal component space, \hat{X}^{TSP} represents a projection of \tilde{X}^{TSP} on a residual space, and \hat{X}^{TSP} and \tilde{X}^{TSP} satisfy satisfy an orthogonal relationship; wherein \hat{X}^{TSP} and \tilde{X}^{TSP} satisfy the orthogonal relationship, wherein the orthogonal relationship is proved as follows:
$$\begin{matrix}{[ {\hat{X}}^{TSP} ]}^{T} {\tilde{X}}^{TSP} = {[ {X}^{TSP} {{U}_{Key}^{{P}_{PCA} \times {P}_{PCA}} [ {U}_{Key}^{{P}_{PCA} \times {P}_{PCA}} ]}^{T} ]}^{T} \cdot \\ {X}^{TSP} ( {I}^{{P}_{PCA} \times {P}_{PCA}} - {{U}_{Key}^{{P}_{PCA} \times {P}_{PCA}} [ {U}_{Key}^{{P}_{PCA} \times {P}_{PCA}} ]}^{T} ) \\ = {{{U}_{Key}^{{P}_{PCA} \times {P}_{PCA}} [ {U}_{Key}^{{P}_{PCA} \times {P}_{PCA}} ]}^{T} [ {X}^{TSP} ]}^{T} {X}^{TSP} ( {I}^{{P}_{PCA} \times {P}_{PCA}} - \\ {{U}_{Key}^{{P}_{PCA} \times {P}_{PCA}} [ {U}_{Key}^{{P}_{PCA} \times {P}_{PCA}} ]}^{T} ) \\ = {{{U}_{Key}^{{P}_{PCA} \times {P}_{PCA}} [ {U}_{Key}^{{P}_{PCA} \times {P}_{PCA}} ]}^{T} [ {X}^{TSP} ]}^{T} {X}^{TSP} {I}^{{P}_{PCA} \times {P}_{PCA}} - \\ {{{U}_{Key}^{{P}_{PCA} \times {P}_{PCA}} [ {U}_{Key}^{{P}_{PCA} \times {P}_{PCA}} ]}^{T} [ {X}^{TSP} ]}^{T} \\ {X}^{TSP} {{U}_{Key}^{{P}_{PCA} \times {P}_{PCA}} [ {U}_{Key}^{{P}_{PCA} \times {P}_{PCA}} ]}^{T}\end{matrix}$$
$$subject to {{U}_{Key}^{{P}_{PCA} \times {P}_{PCA}} [ {U}_{Key}^{{P}_{PCA} \times {P}_{PCA}} ]}^{T} = {I}^{{P}_{PCA} \times {P}_{PCA}} = {[ {X}^{TSP} ]}^{T} {X}^{TSP} - {[ {X}^{TSP} ]}^{T} {X}^{TSP} = 0$$
expressing the drift indicator control limit as:
$${T}_{CL}^{2} = \frac{{P}_{PCA} ( {N}_{TSP} - 1 )}{( {N}_{TSP} - {P}_{PCA} )} {F}_{α} ( {P}_{PCA} , {N}_{TSP} - {P}_{PCA} ) ;$$
$${SPE}_{CL} = {{Θ}_{1} ( \frac{{c}_{α} \sqrt{2 {Θ}_{2} {h}_{0}^{2}}}{{Θ}_{1}} + 1 + \frac{{Θ}_{2} {h}_{0} ( {h}_{0} - 1 )}{{Θ}_{1}^{2}} )}^{1 / {h}_{0}} ;$$
wherein, T_{CL}² is a control limit of Hotelling's T²; SPE_{CL} is a control limit of Squared Prediction Error (SPE); P_{PCA} is a number of selected principal components Fα(P_{PCA}, N_{TSP}−P_{PCA}), Fα(P_{PCA}, N_{TSP}−P_{PCA}) represents an F distribution with degrees of freedom of P_{PCA} and (N_{TSP}−P_{PCA}); c_{α}represents a normal deviation not exceeding (1−α); Θ₁, Θ₂ and h₀ are calculated as follows:
$${h}_{0} = 1 - 2 {Θ}_{1} {Θ}_{3} / 3 {Θ}_{1}^{2} ;$$
$${Θ}_{i} = {\sum }_{m = {F}_{PCA} + 1}^{M} {( {σ}_{m} )}^{i} , i = 1 , 2 , 3 ;$$
wherein, Θ₁, Θ₂ and h₀, are all intermediate variables in SPE control limit calculation, and σₘ is an eigenvalue of singular value decomposition.

4. The online soft measurement method for dioxin emission concentration in the MSWI process according to claim 3, wherein the step of constructing the offline model based on FTBL, and inputting the process data of the typical sample pool and the historical DXN true value data of MSWI into the offline model for prediction calculation to obtain the offline calculation result comprises:
for given TSP data D^{TSP}={xₙ, yₙ}ₙ ^{N}^{_{TSP}}∈ [symbol] ^{N}^{_{TSP}}^{×(M+1)}, randomly selecting a feature value x in D^{TSP} to define node splitting function μ_{CS}^{t}(x), wherein: μ_{CS}^{t}(x)=rand(n,m), n∈(1,N_{TSP}) and m∈(1,M)
wherein μ_{CS}^{t}(x) is a symbolic function, rand(·) is a random number generation function, and n and m do not take maximum and minimum values;
determining K fuzzy rules for Takagi-Sugeno (TS) fuzzy reasoning, wherein a k-th rule is expressed as:
$${R}_{k} : if {x}_{1}^{{t}_{leaf}} is {φ}_{1}^{k} ( {x}_{1}^{{t}_{leaf}} ) and … and {x}_{{M}^{{t}_{leaf}}}^{{t}_{leaf}} is {μ}_{{M}^{{t}_{leaf}}}^{k} ( {x}_{{M}^{{t}_{leaf}}}^{{t}_{leaf}} )$$
$$then {y}^{k} = {g}^{k} ( {x}_{1} , … , {x}_{{M}^{{t}_{leaf}}} )$$
wherein:
$${φ}_{m}^{k} ( {x}_{m}^{{t}_{leaf}} ) = exp [ - {( {x}_{m}^{{t}_{leaf}} - {c}_{k , m} )}^{2} / {σ}_{k , m}^{2} ] ;$$
wherein, Rₖ is a k-th fuzzy rule, c_{k,m} and σ_{k,m} respectively represent a center and a width of a Gaussian function φₘ^{k}(·), t_{leaf}, represents a t_{leaf}-th leaf node; and φₘ^{k}(·) is the Gaussian function;
according to the above K fuzzy rules, describing a result of Fuzzy Decision Tree (FDT) as:
$${\hat{y}}_{i} = f ( {x}_{i} ) = {\sum }_{k = 1}^{K} {\overline{o}}^{k} {g}^{k} ( {x}_{i} )$$
$$wherein :$$
$${\overline{o}}^{k} = {\prod }_{m = 1}^{{M}^{Leaf}} {φ}_{m}^{k} ( {x}_{m}^{{t}_{leaf}} ) {/ \sum }_{i = 1}^{K} ( {\prod }_{m = 1}^{{M}^{Leaf}} {φ}_{m}^{k} ( {x}_{m}^{{t}_{leaf}} ) )$$
$${g}^{k} ( {x}_{i}^{Leaf} ) = {x}_{i}^{Leaf} {ω}_{T S}^{k}$$
wherein, f(·) is an FDT model, and ō^{k} and g^{k}(·) represents an antecedent part and a consequent part of TS fuzzy reasoning, and ω_{TS}^{k} represents a weight of features of the consequent part;
applying a gradient descent method to update parameters during a training process of the FDT model f(·); wherein the parameters comprise the center cₖ, the width σₖ and the weight ω_{TS}^{k}; wherein an output of the feature mapping layer is expressed as follows:
$${Z}_{F M}^{{N}_{F M}} = [ {𝓏}_{F M}^{1} , {𝓏}_{F M}^{2} , … , {𝓏}_{F M}^{{N}_{F M}} ] ∈ {ℝ}^{{N}_{T S P} \times {N}_{F M}}$$
$$where :$$
$${𝓏}_{F M}^{{n}_{F M}} = {f}_{{n}_{F M}}^{F M} ( {X}^{T S P} ) ∈ {ℝ}^{{N}_{T S P} \times 1}$$
wherein, Z_{FM}ⁿ^{_{FM}} is an output of an n_{FM}-th FDT model through an input X^{TSP};
the enhancement layer takes Z_{FM}^{N}^{_{FM}} as input, and an output of the enhancement layer is expressed as:
$${Z}_{E n}^{{N}_{En}} = [ {𝓏}_{E n}^{1} , {𝓏}_{En}^{2} , … , {𝓏}_{E n}^{{N}_{En}} ] ∈ {ℝ}^{{N}_{T S P} \times {N}_{En}} ;$$
an output of the feature mapping layer and the enhancement layer is
$${G}^{{N}_{F M} + {N}_{En}} = [ {Z}_{F M}^{{N}_{F M}} \vert {Z}_{E n}^{{N}_{En}} ] ;$$
using a ridge regression learning algorithm to calculate a weight W^{N}^{_{FM}}^{+N}^{_{En}} between G^{N}^{_{FM}}^{+N}^{_{En}} and a predicted output:
$$\begin{matrix}{W}^{{N}_{F M} + {N}_{En}} = {( λ I + {{G}^{{N}_{F M} + {N}_{En}} ( {G}^{{N}_{F M} + {N}_{En}} )}^{T} )}^{- 1} {( {G}^{{N}_{F M} + {N}_{En}} )}^{T} {y}^{T S P} \\ = {( {G}^{{N}_{F M} + {N}_{En}} )}^{*} {y}^{T S P}\end{matrix} ;$$
wherein, (G^{N}^{_{FM}}^{+N}^{_{En}})* is a pseudo-inverse matrix, λ is a regularization coefficient, and I is an identity matrix;
adding the FDT model in the incremental layer and dynamically updating the pseudo-inverse matrix, wherein Z_{En}^{N}_{En} as input and G^{N}^{_{FM}}^{+N}^{_{En}}⁺¹=[Z_{FM}^{N}^{_{FM}}|Z_{En}^{N}^{_{En}}|z_{In}¹] as output, a pseudo-inverse matrix update process of a incremental process is as follows:
$${[ {G}^{{N}_{F M} + {N}_{En} + 1} ]}^{*} = [ \begin{matrix}{[ {G}^{{N}_{F M} + {N}_{En} + 1} ]}^{*} - {DB}^{T} \\ {B}^{T}\end{matrix} ]$$
$$wherein :$$
$$\{ \begin{matrix}D = {[ {G}^{{N}_{F M} + {N}_{En} + 1} ]}^{*} {H}_{k + 1} \\ {B}^{T} = \{ \begin{matrix}{[ C ]}^{*} , & if C \neq 0 \\ {[ 1 + {D}^{T} D ]}^{- 1} {{D}^{T} [ {G}^{{N}_{F M} + {N}_{En} + 1} ]}^{*} , & if C = 0\end{matrix} \\ C = {H}_{k + 1} - {G}^{{N}_{F M} + {N}_{En} + 1} D\end{matrix}$$
wherein, D, Hₖ₊₁, B^{T} and C are all intermediate variables in the pseudo-inverse matrix update process;
wherein a new weight matrix W^{N}^{_{FM}}^{+N}^{_{En}}⁺¹ is expressed as:
$${W}^{{N}_{F M} + {N}_{E n} + 1} = [ \begin{matrix}{W}^{{N}_{F M} + {N}_{E n}} - D {B}^{T} {y}^{T S P} \\ {B}^{T} {y}^{T S P}\end{matrix} ] ;$$
wherein a prediction calculation process of the offline model FTBL is as follows:
$$\hat{y} = {G}^{{N}_{F M} + {N}_{En} + 1} {W}^{{N}_{F M} + {N}_{En} + 1} .$$

5. The online soft measurement method for dioxin emission concentration in the MSWI process according to claim 4, wherein principal component analysis is performed based on the online data, and whether the online data is judged to be the drift data or the normal data based on the drift indicator control limit, when the online data is the normal data, jump to step “constructing an offline model based on FTBL, and inputting the process data of the typical sample pool and historical DXN true value data of MSWI into the offline model for prediction calculation to obtain an offline calculation result”; when the online data is the drift data, the online model based on FTBL is constructed, the process data of the typical sample pool, the drift data and the output data of the incremental layer of the offline model are input into the online model for prediction calculation to obtain the online calculation result, comprising:
calculating a drift value within a new window based on:
$${T}_{{O}_{1}}^{2} = {x}_{{O}_{1}}^{T} {U}_{{N}_{T S P} + 1}^{{P}_{P C A} \times {P}_{P C A}} {\sum }_{{N}_{T S P} + 1}^{- 1} {( {U}_{{N}_{T S P} + 1}^{{P}_{P C A} \times {P}_{P C A}} )}^{T} {x}_{{O}_{1}} ;$$
$$SP {E}_{{O}_{1}} = {x}_{{N}_{T S P} + 1}^{T} ( I - {{U}_{{N}_{T S P} + 1}^{{P}_{P C A} \times {P}_{P C A}} ( {U}_{{N}_{T S P} + 1}^{{P}_{P C A} \times {P}_{P C A}} )}^{T} ) {x}_{{N}_{T S P} + 1} ;$$
wherein, T_{O}_{₁}² and SPE_{O}_{₁} are statistical indicators of (N_{TSP}+1)-th process x_{O}_{₁} data, U_{N}_{_{TSP}}₊₁^{P}^{_{PCA}}^{×P}^{_{PCA}} represent a new load matrix, and Σ_{N}_{_{TSP}}₊₁⁻¹ represent a new diagonal matrix;
determining whether the sample is a drift sample or a normal sample through a judgment formula; wherein the judgment formula is:
$$\{ \begin{matrix}drift & if {T}_{{O}_{1}}^{2} > {T}_{CL}^{2} and {SPE}_{{O}_{1}} > {SPE}_{C L} \\ normal & etc .\end{matrix}$$
for normal samples, the offline model of FTBL is reused for soft measurement of DXN concentration, wherein the offline model of FTBL is expressed as:
$${\hat{y}}_{{O}_{t}} = {G}^{k + {N}_{In}} {W}^{k + {N}_{In}}$$
$$= [ {Z}_{F M}^{{N}_{F M}} \vert {Z}_{E n}^{{N}_{En}} \vert {Z}_{In}^{{N}_{In}} ] {W}^{k + {N}_{In}}$$
$$= [ {f}_{1}^{F M} ( {x}_{{O}_{t}} ) , … , {f}_{{N}_{F M}}^{F M} ( {x}_{{O}_{t}} ) \vert {f}_{1}^{E n} ( {x}_{{O}_{t}} ) , … , {f}_{{N}_{En}}^{E n} ( {x}_{{O}_{t}} ) \vert {f}_{1}^{I n} ( {x}_{{O}_{t}} ) , … , {f}_{{N}_{I n}}^{I n} ( {x}_{{O}_{t}} ) ] {W}^{k + {N}_{In}}$$
for drift samples, a soft measurement value is calculated as:
$${\hat{y}}_{{O}_{t}} = {G}^{k + {N}_{In}} {W}^{k + {N}_{In}} + {ε}_{Offset}$$
wherein, ε_{Offset} is an offset value of an offline FTBL prediction output is as follows:
$${ε}_{Offset} = \{ \begin{matrix}+ \frac{1}{{N}_{t}} \sum ( {\hat{y}}_{t} - E ( \hat{y} ) ) , if {x}_{IncTem} < E ( {x}_{IncTem} ) \\ - \frac{1}{{N}_{t}} \sum ( {\hat{y}}_{t} - E ( \hat{y} ) ) , if {x}_{IncTem} \geq E ( {x}_{IncTem} )\end{matrix}$$
wherein, Nₜ represents a total amount of data arriving at time t, ŷ represents all predicted values arriving at time t, E(ŷ) represents a mathematical expectation of a vector ŷ, and X_{IncTem} represents an incinerator temperature at time t;
when a true value is detected, the TSP data, drift data and the output of the incremental layer are input into the online model; wherein the predicted value of the online model is:
$${\hat{y}}_{{O}_{t}} = {G}^{{N}_{F M} + {N}_{En} + {N}_{I n} + {N}_{O I}} {W}^{{N}_{F M} + {N}_{En} + {N}_{I n} + {N}_{O I}}$$
wherein, W^{N}^{_{FM}}^{+N}^{_{En}}^{+N}^{_{In}}^{+N}^{_{OI}} represents a weight matrix, and G^{N}^{_{FM}}^{+N}^{_{En}}^{+N}^{_{In}}^{+N}^{_{OI}} is an FDT output matrix of N_{FM}+N_{En}+N_{In}+N_{OI}.
